(19) United States
(12) Patent Application Publication
(10) Pub. No.: US 2023/0201837 A1
(43) Pub. Date: Jun. 29, 2023

(54) SYSTEM, DEVICE, AND METHODS FOR TESTING

(71) Applicant: NICOYA LIFESCIENCES, INC., Kitchener (CA)

(72) Inventors: Ryan DENOMME, Kitchener, Ontario (CA); Michael POLLACK, Durham, NC (US); Arjun SUDARSAN, Carlsbad, CA (US); Harish KRISHNAKUMAR, Mississauga, Ontario (CA)

(21) Appl. No.: 17/996,815

(22) PCT Filed: Apr. 23, 2021
(86) PCT No.: PCT/CA2021/050563

§ 371 Date: Oct. 21, 2022

Related U.S. Application Data
(60) Provisional application No. 63/014,629, filed on Apr. 23, 2020.
(60) Provisional application No. 63/074,068, filed on Sep. 3, 2020.
(60) Provisional application No. 63/123,594, filed on Dec. 10, 2020.

(51) Int. Cl. B01L 3/00; G01N 21/78; G01N 33/569; G01N 33/543

(52) U.S. Cl. CPC B01L 3/502792; G01N 21/78; G01N 33/54346; G01N 33/56983; B01L 2200/16; B01L 2300/023; B01L 2300/024; B01L 2300/0645; G01N 2333/165

(57) ABSTRACT

The present disclosure provides a digital microfluidic (DMF) cartridge for performing a self-test for a target analyte, including a DMF cartridge comprising a bottom substrate and a top substrate separated by a droplet operations gap, wherein the bottom substrate comprises a plurality of droplet operations electrodes configured for performing droplet operations on a liquid droplet in the droplet operations gap; one or more reaction chambers or reaction zones on the bottom substrate that are supplied by an arrangement of the droplet operations electrodes, wherein each reaction chamber or reaction zone comprises at least one detection spot and is configured for performing a plasmonic particle-assisted ELISA (pELISA) for detection and quantification of a target analyte in a sample droplet. The device may include downloadable software for a self-test and be operable using a smart device.

### CROSS-REFERENCE TO RELATED APPLICATIONS

This application claims priority to U.S. Provisional Pat. App. No. 63/123,594 entitled “POINT-OF-CARE (POC) SYSTEM, DEVICE, AND METHODS SUITABLE FOR PERFORMING BIOLOGICAL ANALYSIS IN A SELF-TEST ENVIRONMENT” filed on Dec. 10, 2020, U.S. Provisional Pat. No. 63/074,068 entitled “TESTING SYSTEM, DEVICE AND METHOD” filed on Sep. 3, 2020, and U.S. Provisional Pat. No. 63/014,629 entitled “TESTING SYSTEM, DEVICE AND METHOD” filed on Apr. 23, 2020, each of which is incorporated by reference herein.

### FIELD

The present disclosure relates to the analysis of biological materials and more particularly to a point-of-care (POC) system, device, and methods suitable for performing a test, such as a self-test.

### BACKGROUND

The COVID-19 pandemic has highlighted the need for novel, user-friendly test devices that can be used to rapidly test for infectious diseases. There is also a need for a device that can be used by non-professionals, including by individuals to test themselves. There is also a need for a device that can gather public health information from geographically distributed testing locations.

### SUMMARY

The present disclosure provides a digital microfluidic (DMF) device for performing a self-test for a target analyte. The DMF device may include a DMF cartridge comprising a bottom substrate and a top substrate separated by a droplet operations gap, wherein the bottom substrate may include a plurality of droplet operations electrodes configured for performing droplet operations on a liquid droplet in the droplet operations gap. The DMF device may include one or more reaction chambers or reaction zones on the bottom substrate that are supplied by an arrangement of the droplet operations electrodes, wherein each reaction chamber or reaction zone may include at least one detection spot and is configured for performing a plasmonic particle-assisted ELISA (pELISA) for detection and quantification of a target analyte in a sample droplet. The DMF device may include a controller coupled to the electrodes and programmed to activate and deactivate the electrodes and thereby effect droplet operations for performing the self-test.

The bottom substrate may include a printed circuit board. The bottom substrate may include one or more reservoir electrodes configured for supplying the one or more reaction chambers or reaction zones via the droplet operations electrodes.

The top substrate may include a glass or plastic substrate that is substantially transparent to light. The top substrate may include one or more input ports for receiving and supplying an input reagent or sample fluid, wherein the input ports are arranged in relation to the one or more reservoir electrodes on the bottom substrate. The top substrate may include one or more reagent wells for receiving a reagent blister pack, wherein the reagent wells are arranged in relation to the one or more reagent reservoir electrodes on the bottom substrate.

The one or more reagent wells may include a reagent port arranged to permit flow of reagent fluids from a reagent blister pack into the well. The reagent well may include a two-port well. The two-port well may include a first input port comprising a luer port or simple port well for receiving and inputting a sample fluid; and a second input port comprising a reagent blister pack well for receiving and inputting a reagent fluid. The second input port may include a blister pack burst mechanism attached to the top plate in proximity to the input second port for bursting a reagent blister pack and releasing the reagent fluids. The blister pack burst mechanism may include a pointed or sharp-edged feature. The two-port well may include two sample input ports for receiving and inputting a sample fluid.

The top or bottom substrate may include one or more detection spots arranged in relation to the one or more reaction chambers and/or reaction zones on the bottom substrate for positioning a droplet for detection.

The device may include one or more thermal control mechanisms situated in sufficient proximity of the droplet operations gap to permit thermal control in the droplet operations gap for controlling the processing temperature in the DMF device.

The device may include one or more magnets situated in sufficient proximity to the droplet operations gap to permit magnetic manipulation of magnetically responsive beads and/or particles in a droplet in the droplet operations gap.

The device may include a power source electrically coupled to the plurality of droplet operations electrodes in the droplet operations gap for supplying power for performing droplet operations on a liquid droplet in the droplet operations gap.

The power source may include a wired communications link. The wired communications link may include a USB charging cable of a smart device.

The device may include communications interface for electronically connecting electronic components of the device to the controller and exchanging test information from the at least one detection spot with a remote computer processing unit (CPU). The controller and/or the remote CPU may be part of a smart device. The communications interface may include a wired and/or wireless communication interface. The device may include computer memory for storing self-test information.

The present disclosure provides a system for performing a self-test for a target analyte. The system may include any DMF device including memory and a self-test application loaded in the memory for downloading onto a smart device. The self-test application may provide a user interface for operating the system and/or the DMF device and instructions for performing a pELISA test for a target analyte.

The self-test application may include an algorithm for processing digital image data of the pELISA test to produce a colorimetric readout based on a colorimetric change. The self-test application may include an algorithm for analyzing the colorimetric readout to determine the presence or absence of a target analyte.

The user interface may include a display for presenting the results of the self-test to the user.

The digital image data may include image data captured using an image capture device operated by the user. The user’s image capture device may include an on-board camera of the user’s smart device. Captured image data is stored in memory on the user’s smart device. The system may include a communications link for providing a communication path between the DMF device and the user’s smart device. The system may include data storage associated with a networked computer via a network for storing and sharing the self-test information.

The present disclosure provides a method of performing an analysis for a target analyte. The method may include providing a DMF device or cartridge of the present disclosure. The method may include providing a reaction surface and a capture molecule in the one or more reaction chambers or reaction zones in the droplet operations gap of the DMF device. The method may include using droplet operations effected by the controller to introduce a sample fluid onto the reaction surface, wherein the sample fluid potentially may include a target analyte that binds to the capture molecule, forming a target-capture molecule complex immobilized on the reaction surface. The method may include using droplet operations effected by the controller to introduce a droplet including a detection antibody onto the reaction surface. An enzyme may thus be conjugated to the detection antibody and/or a capture molecule. The method may include using droplet operations effected by the controller to introduce a detection solution comprising an enzyme substrate onto the reaction surface, wherein in the presence of a target-capture molecule complex a colorimetric change is produced. The method may include measuring at the one or more detection spots in each of the one or more reaction chambers or reaction zones the colorimetric change in response to the enzyme catalyzed detection of the target analyte.

In some embodiments, the reaction surface may include a plasmonic nanoparticle immobilized thereon and the capture molecule is suspended in a solution on the reaction surface. In some embodiments, the reaction surface may include a plasmonic nanoparticle and a capture molecule immobilized thereon. In some embodiments, the reaction surface may include the capture molecule immobilized thereon, and the detection solution may include a plasmonic nanoparticle. In some embodiments, the plasmonic nanoparticle may include a nanosphere, a nanorod, a nanourchin, or a nanostar. In some embodiments, the plasmonic nanoparticle may include two or more types of plasmonic nanoparticles, thereby increasing the sensitivity and/or range of detection for a target analyte. In some embodiments, the plasmonic nanoparticle may include a gold nanoparticle. In some embodiments, the gold nanoparticle may include a gold nanosphere and/or a gold nanourchin. In some embodiments, the reaction surface may include a substrate surface of the DMF device. In some embodiments, the reaction surface may include a magnetically responsive bead. In some embodiments, the capture molecule may include an antibody. In some embodiments, the capture molecule may include an antigen. In some embodiments, the sample fluid may include a bodily fluid from a human or an animal. In some embodiments, the target analyte may include two or more target analytes. In some embodiments, the target analyte is a protein. In some embodiments, the protein is an antibody. In some embodiments, the antibody is an IgG or IgM antibody. In some embodiments, the target analyte is a molecule or molecular structure from a virus, a bacterium, or any other pathogen. In some embodiments, the target analyte may include a molecule or molecular structure bound to the outer surface of a virus, a bacterium, or any other pathogen. In some embodiments, the target analyte may include a molecule or molecular structure that is internal to a virus, a bacterium, or any other pathogen. In some embodiments, the internal molecule or molecular structure is exposed by disrupting the integrity of the virus, the bacterium, or any other pathogen. In some embodiments, the detection antibody may include a primary antibody conjugated to an enzyme. In some embodiments, the detection antibody may include a secondary antibody conjugated to an enzyme. In some embodiments, the enzyme may include horseradish peroxidase (HRP). In some embodiments, the enzyme substrate may include TMB. In some embodiments, the detection solution may include a metal ion precursor. In some embodiments, the detection solution may include a fluorescent probe. In some embodiments, the colorimetric change may include a change in the intensity of a color and/or perceivable color hue. In some embodiments, the colorimetric change is caused by etching of the plasmonic nanoparticle in response to the enzyme catalyzed detection of the target analyte. In some embodiments, the colorimetric change is caused by aggregation of the plasmonic nanoparticle in response to the enzyme catalyzed detection of the target analyte. In some embodiments, the colorimetric change is caused by growth of the plasmonic nanoparticle in response to the enzyme catalyzed detection of the target analyte. In some embodiments, the colorimetric change is caused by quenching and/or unquenching the fluorescence of a fluorescent probe in response to the enzyme catalyzed detection of the target analyte.

In some embodiments, measuring the colorimetric change may include capturing a digital image of the colorimetric changes at each detection spot of the one or more reaction chambers or reaction zones; processing the digital image data to produce a colorimetric readout based on the colorimetric change; and analyzing the colorimetric readout to determine the presence or absence of the target analyte. In some embodiments, processing the digital image data may include using a color-based detection algorithm to produce the colorimetric readout. In some cases, analyzing the colorimetric readout may include using an algorithm to differentiate a positive or a negative sample based on the colorimetric results.

In some cases, the methods of the present disclosure include concentrating the target analyte prior to analysis.

The present disclosure provides a method of performing a user conducted self-test for a target analyte. The method may include: downloading the self-test application onto the user’s smart device to initiate and set up the self-testing process; introducing a user sample into one or more sample reservoirs of the DMF device, wherein the pELISA test is automatically performed to test for the presence or absence of the target analyte; capturing a digital image of the pELISA test results for automated analysis for determining the presence or absence of the target analyte; and conducting the automated analysis.

In some embodiments, setting up the self-testing process may include establishing a communication link between the user’s smart device and the DMF device; and capturing an image of a QR code provided on the DMF device and collecting any other required test information.

In some embodiments, the user sample may include a saliva sample. In some cases, the methods of the present disclosure include presenting the self-test results to the user. In some cases, the methods of the present disclosure include sharing the results of the self-test with a networked computer. In some cases, the methods of the present disclosure include stopping the self-testing processes if the user decides that he/she are not ready to continue the testing process. In some cases, the methods of the present disclosure include introducing an assay buffer and detection solution into one or more reagent wells of the DMF device.

### BRIEF DESCRIPTION OF DRAWINGS

The features and advantages of the present disclosure will be more clearly understood from the following description taken in conjunction with the accompanying drawings, which are not necessarily drawn to scale, and wherein:

FIG. 1 illustrates a block diagram of an example of the system and test cartridge suitable for performing test, such as a self-test;

FIG. 2A, FIG. 2B, and FIG. 2C illustrate a perspective view, an exploded view, and a cross-sectional view of an example of the basic structure forming the DMF portion of the test cartridge;

FIG. 3 shows an example implementation of the system and test cartridge suitable for performing test, such as a self-test;

FIG. 4 illustrates a schematic diagram of an example of a pELISA assay performed at the pELISA-based immunoassay panel of the presently disclosed of the system and test cartridge;

FIG. 5 illustrates a flow diagram of an example of a method of performing an pELISA assay on the system and test cartridge;

FIGS. 6A through 6F illustrate pictorially an example of certain steps in the method of operation shown in FIG. 5;

FIG. 7 illustrates an example of an image analysis histogram generated using the system and test cartridge;

FIG. 8 shows a photo of an example of a test cartridge layout that may be representative of the test cartridge;

FIG. 9A and FIG. 9B show photos of an example of the test cartridge and showing the DMF-portion thereof;

FIG. 10 shows another implementation of the system and test cartridge suitable for performing test, such as a self-test;

FIG. 11 illustrates a flow diagram of an example of a method of using of the system and test cartridge;

FIGS. 12A through 12E illustrate pictorially an example of the method of use shown in FIG. 11;

FIG. 13 shows another photo of the test cartridge shown in FIG. 9A and FIG. 9B;

FIG. 14 illustrates a perspective view of another example of the test cartridge of the system;

FIG. 15, FIG. 16, and FIG. 17 illustrate a top view, a side view, and a bottom view, respectively, of the test cartridge shown in FIG. 14;

FIG. 18 illustrates a plan view of an example of the PDB bottom substrate of the test cartridge shown in FIG. 14;

FIG. 19 illustrates a perspective view of the portion of the test cartridge shown in FIG. 14 that includes the three-reagent blister pack wells;

FIG. 20 illustrates a cross-sectional view of the test cartridge taken along line A-A of FIG. 14;

FIGS. 21 through 26 illustrate cross-sectional views of various portions, respectively, of the test cartridge shown in FIG. 14; and

FIG. 27 is a plot showing the immobilization of the SARS-CoV-2 spike protein RBD on an OpenSPR-XT carboxyl sensor;

FIG. 28 is a plot showing the binding and kinetic fits of the SARS-CoV-2 primary antibody to the immobilized receptor domain (i.e., spike protein RBD);

FIG. 29 is a plot showing the binding and kinetic fits of the rabbit serum diluted SARS-CoV-2 primary antibody to the immobilized receptor domain (i.e., spike protein RBD);

FIG. 30 is a plot showing a primary antibody plus secondary antibody amplification cycle;

FIG. 31A is a plot showing an example of an SPR assay result for the R001 antibody;

FIG. 31B is a plot showing the epitope overlap for the MM57 and MM42 antibodies;

FIG. 32 is a plot showing a representative example of a cross reactivity study using MM57 and MM42 antibodies against SARS-CoV-1 spike protein using SPR;

FIG. 33 is a plot showing the absorbance readout at 450 nm for the ELISA plate assay using R001 to capture spike protein diluted into either buffer or saliva;

FIG. 34A is a plot showing sub 100 pM detection of biotinylated spike protein captured with streptavidin coated magnetically responsive beads;

FIG. 34B is a plot showing sub 100 pM detection of spike protein using R001 coated magnetically responsive beads to capture spike protein;

FIG. 35 is a plot showing the correct identification of all saliva samples as either positive or negative for spike protein;

FIG. 36A is a plot showing the results from the magnetic bead ELISA on inactive SARS-CoV-2 virus;

FIG. 36B is a photo of the ELISA results of FIG. 37A showing that all three concentrations of virus could be visually detected relative to the control sample;

FIG. 37 is a panel of plots 2300 showing UV-Vis spectra of nanoruchins exposed to different concentrations of oxidized TMB+ or TMB2+ with 5 mM CTAB;

FIG. 38 is photographs and UV-Vis spectral plots of gold nanourchins (AuNU) exposed to TMB+ and TMB2+ after 3 and 10 minutes of incubation; and

FIG. 39 is a photograph showing a comparison between the plasmonic ELISA and a conventional ELISA result over a range of oxidized TMB concentrations.

### DETAILED DESCRIPTION

### Terminology

“Activate” with reference to one or more electrodes means effecting a change in the electrical state of the one or more electrodes which results in a droplet operation.

“Controller” means a hardware processor, hardware controller, or other chip, circuit or device having the capability of processing digital instructions. A controller may be electronically coupled to switches for controlling electrode activation, sensors, and other electronic components.

“Droplet” means a volume of liquid on a test cartridge that is at least partially bounded by filler fluid. For example, a droplet may be completely surrounded by filler fluid or may be bounded by filler fluid and one or more surfaces of the test cartridge. Droplets may, for example, be aqueous or non-aqueous or may be mixtures or emulsions including aqueous and non-aqueous components. Droplets may take a wide variety of shapes; nonlimiting examples include generally disc shaped, slug shaped, truncated sphere, ellipsoid, spherical, partially compressed sphere, hemispherical, ovoid, cylindrical, and various shapes formed during droplet operations, such as merging or splitting or formed as a result of contact of such shapes with one or more surfaces of a test cartridge.

“Droplet operation” means any manipulation of a droplet on a test cartridge. A droplet operation may, for example, include: loading a droplet into the test cartridge; dispensing one or more droplets from a source droplet; splitting, separating or dividing a droplet into two or more droplets; transporting a droplet from one location to another in any direction; merging or combining two or more droplets into a single droplet; diluting a droplet; mixing a droplet; agitating a droplet; deforming a droplet; retaining a droplet in position; incubating a droplet; heating a droplet; vaporizing a droplet; condensing a droplet from a vapor; cooling a droplet; disposing of a droplet; transporting a droplet out of a test cartridge; other droplet operations described herein; and/or any combination of the foregoing. The terms “merge,” “merging,” “combine,” “combining” and the like are used to describe the creation of one droplet from two or more droplets. It should be understood that when such a term is used in reference to two or more droplets, any combination of droplet operations sufficient to result in the combination of the two or more droplets into one droplet may be used. For example, “merging droplet A with droplet B,” can be achieved by transporting droplet A into contact with a stationary droplet B, transporting droplet B into contact with a stationary droplet A, or transporting droplets A and B into contact with each other. The terms “splitting,” “separating” and “dividing” are not intended to imply any particular outcome with respect to size of the resulting droplets (i.e., the size of the resulting droplets can be the same or different) or number of resulting droplets (the number of resulting droplets may be 2, 3, 4, 5 or more). The term “mixing” refers to droplet operations which result in more homogenous distribution of one or more components within a droplet. Examples of “loading” droplet operations include microdialysis loading, pressure assisted loading, robotic loading, passive loading, and pipette loading. In various embodiments, the droplet operations may be electrode mediated, e.g., electrowetting mediated or dielectrophoresis mediated. For examples of electrowetting architectures suitable for conducting droplet operations with the present disclosure, see U.S. Pat. No. 6,911,132, entitled “Apparatus for Manipulating Droplets by Electrowetting-Based Techniques,” issued on Jun. 28, 2005 to Pamula et al.; U.S. Pat. Application Ser. No. 11/343,284, entitled “Apparatuses and Methods for Manipulating Droplets on a Printed Circuit Board,” filed on Jan. 30, 2006; U.S. Pat. No. 6,773,566, entitled “Electrostatic Actuators for Microfluidics and Methods for Using Same,” issued on Aug. 10, 2004 and U.S. Pat. No. 6,565,727, entitled “Actuators for Microfluidics Without Moving Parts,” issued on Jan. 24, 2000, both to Shenderov et al.; Pollack et al., International Patent Application No. PCT/US2006/047486, entitled “Droplet-Based Biochemistry,” filed on Dec. 11, 2006, the disclosures of which are incorporated herein by reference. Methods of the disclosure may be executed using droplet actuator systems, e.g., as described in International Patent Application No. PCT/US2007/009379, entitled “Droplet manipulation systems,” filed on May 9, 2007. In various embodiments, the manipulation of droplets by a droplet actuator may be electrode mediated, e.g., electrowetting mediated or dielectrophoresis mediated. Electrowetting electrodes may be controlled by a device, such as a computing device, such as a smart device by electronically coupling the device to switches controlling electrical supply to the electrowetting electrodes. A test cartridge may be a droplet actuator.

“Enzyme-linked immunosorbent assay (ELISA)” means an analytical biochemistry assay that is commonly used to measure biological targets, such as antibodies, antigens, proteins, and glycoproteins. ELISA uses a solid-phase type of enzyme immunoassay (EIA) to detect the presence of a ligand (commonly a protein) in a liquid sample using antibodies directed against the protein to be measured. In the simplest form of an ELISA, antigens from the sample to be tested are attached to a surface. Then, a matching antibody is applied over the surface so it can bind the antigen. This antibody is linked to an enzyme and then any unbound antibodies are removed. In the final step, a substance containing the enzyme’s substrate is added. If there was binding the subsequent reaction produces a detectable signal, most commonly a color change.

“Electrically connected,” “electrical connection,” “electrically coupled,” and the like are intended to refer to a connection that is capable of transmitting electricity.

“Electronically connected,” “electronic connection,” “electronically coupled” and the like are intended to include both wired and wireless connections, including without limitation connections that are capable of transmitting data signals, e.g., electrical signals, electromagnetic signals, wireless signals, or optical signals.

“Filler fluid” means a fluid associated with a droplet operations substrate of a test cartridge, which fluid is sufficiently immiscible with a droplet phase to render the droplet phase subject to electrode-mediated droplet operations. The filler fluid may, for example, be a low-viscosity oil, such as silicone oil. Other examples of filler fluids are provided in International Patent Application No. PCT/US2006/047486, entitled, “Droplet-Based Biochemistry,” filed on Dec. 11, 2006; and in International Patent Application No. PCT/US2008/072604, entitled “Use of additives for enhancing droplet actuation,” filed on Aug. 8, 2008.

“pELISA” means plasmonic nanoparticle assisted ELISA or plasmonic ELISA. An example of plasmonic nanoparticle assisted ELISA is provided with reference to the U.S. Pat. App. No. 63/104,006, entitled “Methods of plasmonic nanoparticle assisted enzyme-linked immunosorbent assay (ELISA) in a fluidics device,” filed on Oct. 22, 2020; the entire disclosure of which is incorporated herein by reference.

“Nanoparticles (NP)” means beads or particles with one or more dimensions, e.g., a cross-section, less than about 300 nm.

“Plasmonic nanoparticles” means nanoparticles whose electron density can couple with electromagnetic radiation of wavelengths larger than the particle. Plasmonic nanoparticles exhibit intense light absorbance, scattering, and/or extinction properties. Plasmonic nanoparticles typically consist of at least one layer or component of noble metals (e.g., gold, silver, palladium, platinum, etc.).

“Sample” or “sample fluid” means fluid that is tested using the fluidics device for detection and quantification of target antibodies. The fluid may be artificially spiked with target antibodies and/or other constituents. The fluid may also be collected from humans or animals, such as sweat, saliva, blood, urine, mucous, tear fluid, etc.

“Self-test” or “self-testing” means the user’s sample is tested and the user runs the test, e.g., a user obtains sample from the user, loads the user’s sample onto a test device, runs the test, and obtains the result.

“Smart device” means a computing device that may be electronically coupled to a device of the present disclosure to control the execution of a method or assay of the disclosure. Examples include, but are not limited to, desktop computers, laptop computers, tablet computers, video monitors, televisions, digital video disc players, media streaming devices, smartphones, electronic readers, and video game devices.

“Software” includes firmware, operating systems, applications (e.g., mobile apps) and other types of software. Software may, for example, be written to execute a test, such as a self-test, on the test cartridge of the present disclosure.

“Test” means a biological or chemical assay.

“User” means an individual who is operating a device to control a test cartridge. The present disclosure enables a user to obtain the user’s own sample, load the user’s own sample onto a test device of the present disclosure, run the test and obtain the result. In other words, the user may be the subject of the test. A user may also be a person other than the test subject, such as a laboratory technician.

The terms “top” and “bottom” are used throughout the description with reference to the top and bottom substrates of the test cartridge for convenience only, since the test cartridge is functional regardless of its position in space.

Headings are included herein for reference and to aid in locating the various sections. These headings are not intended to limit the scope of the concepts described with respect to the headings. Such concepts may have applicability throughout the present specification.

Although the present disclosure describes some detail by way of illustration and example for purposes of clarity and understanding, it will be apparent to those skilled in the art that certain changes and modifications may be practiced. Reference to “the invention” or the like is intended as a reference to any of a wide variety of embodiments of, or aspects of, the present disclosure, and not as limiting the present disclosure to a single embodiment or aspect.

The description and examples should not be construed as limiting the scope of the present disclosure to the embodiments and examples described herein, but rather as encompassing all modifications and alternatives falling within the true scope and spirit of the present disclosure.

### Description of the Embodiments

The present disclosure provides a system, test cartridge, and methods suitable for performing biological analysis. In some embodiments, the present disclosure provides a point-of-care (POC) system, test cartridge, and methods suitable for performing test, such as a self-test. In some embodiments, the system, test cartridge, and methods may be used to test for a variety of different biomarkers, such as viral biomarkers, e.g., viral antigens and antibodies. Test functionality can include testing of both active and prior viral infections.

In some embodiments, the system, test cartridge, and methods provide a rapid, low-cost and user-friendly test cartridge for at-home testing of one or more viruses, such as SARS-CoV-2. The present disclosure provides in some embodiments, a single-use disposable test cartridge. The test cartridge may be capable of providing a rapid lab-quality result, e.g., in under 20 minutes. The test may be provided using a self-collected saliva sample. The test may be self-run by the individual user who collected the self-collected sample. The test may execute using droplet operations controlled by a device, such as a computing device, such as a smart device.

In one embodiment, a colorimetric immunoassay (pELISA) may be performed in a test cartridge controlled by a handheld smart device using a digital microfluidics (DMF) liquid-handling technology that automates the assay. Paired with or electronically coupled with a smart device, such as a smartphones, the system, test cartridge, and methods are capable of leveraging cloud technology to interpret and upload results to central databases for efficient monitoring and action.

The test cartridge may include, for example, a printed circuit board (PCB) with an array of droplet operations electrodes. The PCB may be covered by a plastic top substrate that provides access for loading samples and reagents. In some embodiments drive electronics for the test cartridge are integrated on the same PCB as the droplet operations electrodes.

A smart device may be electronically coupled to the test cartridge, e.g., to the droplet operations electrodes, sensors, and other components. The smart device may be used for control, detection, analysis, communications and power, and thereby removing the need for any other equipment. In some embodiments, the system, test cartridge, and methods provide a DMF-based test cartridge that may include a DMF cartridge portion (e.g., a PCB bottom substrate and a plastic top substrate separated by a gap) and a control electronics PCB integrated together in one assembly. In some embodiments, the system, test cartridge, and methods provide a fully functioning DMF-based test cartridge and testbed including magnetic actuation, sample inlet ports that interface with off-the-shelf saliva collection tubes, pockets to store prepackaged reagent blisters and corresponding mechanisms to puncture these blisters at the time of use, and built-in lenses to amplify the color change readout for interpretation of the results of the assay.

In some embodiments, the system, test cartridge, and methods provide a rapid (e.g., less than 30 min, or less than 25 min, or about 20-min from sample to answer) saliva-based pELISA COVID-19 diagnostic test that is able to detect SARS-CoV-2 within the first 72 hours of symptom onset.

In some embodiments, the system, test cartridge, and methods may be portable, disposable, uniquely identifiable, affordable, and requires no specialized training or equipment.

The test cartridge may be electronically coupled to, or paired with, a smart device, such as a smartphone. Test results may be easily interpreted in non-traditional clinical environments or in at home settings, and uploadable to public health databases through the cloud.

In some embodiments, the system, test cartridge, and methods utilize three additional technologies to enhance the detection of SARS-CoV-2 to diagnose individuals within 72 hours of the onset of symptoms- (1) a state of the art plasmonic ELISA (pELISA) immunoassay increases the sensitivity of the colorimetric result by using the etching of gold nanoparticles; (2) the use of magnetically responsive beads combined with DMF facilitates high amplification and concentration of viral particles; and (3) the smartphone application applies an algorithm to efficiently differentiate positive and negative samples based on colorimetric results.

Features of certain aspects of the system, test cartridge, and methods may include, but are not limited to, the following:
(1) Assay in spiked saliva thereby not requiring a nasopharyngeal swab;
(2) <20-minute test with results interpreted visually and/or by a standard smartphone camera;
(3) Limit of Detection (LOD) demonstrated to be less than 12,000 viral copies/mL with a clear path to achieve 5,000 viral copies/mL or lower;
(4) Assay sensitivity and specificity were both measured to be 100% in a small-scale study; and
(5) Selection of an easy-to-use Food and Drug Administration (FDA)-approved saliva collection kit that interfaces with the system, test cartridge, and methods.

In some embodiments, the system, test cartridge, and methods provide a color-based detection means with respect to determining the presence or absence of a virus (e.g., SARS-CoV-2).

In some embodiments, the system, test cartridge, and methods provide means by which a pELISA-based immunoassay panel may be photographed using a smart device (e.g., smartphone), and then the photograph analyzed by a mobile app on the smart device. The mobile app may be used to ascertain whether the virus (e.g., SARS-CoV-2) is present or not. For example, a simple mobile app may be used to analyze the color values (e.g., RGB values) of the pixels and determine the presence or absence of the virus based on these values. For example, a certain set of RGB values may trigger a positive result while other RGB values may trigger a negative result.

In some embodiments, the system, test cartridge, and methods provide a DMF cartridge portion that may include a two-port well or reservoir and wherein one port of the two-port well may be a Luer port, or simple well-port and the second port may be a blister pack and wherein the two-port well may include a blister pack burst mechanism.

### System and Test Cartridge

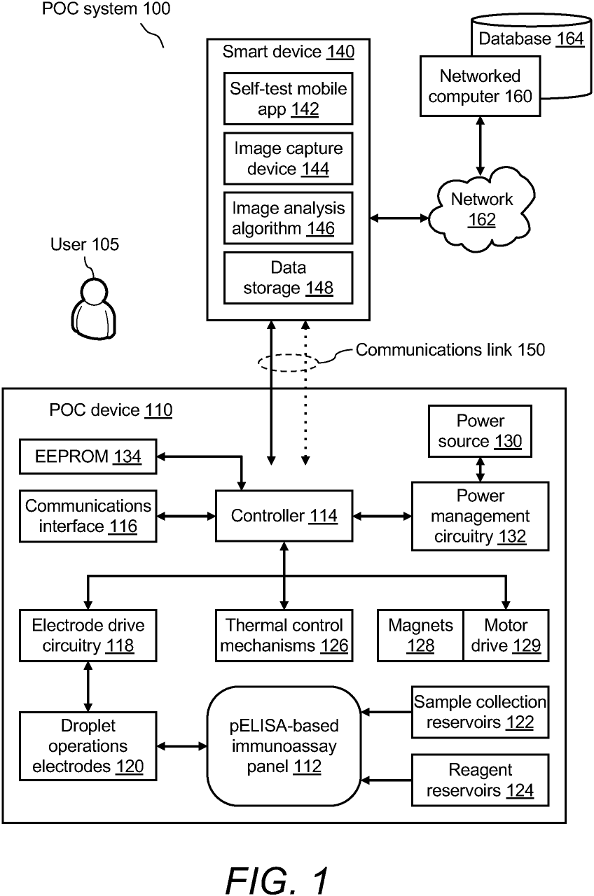

FIG. 1 is a block diagram of an example of the system and test cartridge of the present disclosure. The embodiment illustrated is suitable for performing test, such as a self-test. For example, a system 100 is provided that may include a test cartridge 110 that may be operated via a smart device 140 for performing a test, such as a self-test. That is, a user 105 of system 100 may be the subject of the test. In one example, user 105 may use system 100 for testing, e.g., self-testing, for SARS-Cov-2 viral infection, which is the causative agent of COVID-19. Accordingly, in this example, test cartridge 110 may be a handheld test cartridge 110 may be used to collect a sample from user 105 and then perform the biological analysis with respect to determining the presence or absence of the SARS-Cov-2 antigen in the sample. In system 100, user 105 may use his/her own smart device 140 to operate test cartridge 110. System 100 and test cartridge 110 provide a color-based detection means with respect to determining the presence or absence of a virus (e.g., SARS-CoV-2).

Further, test cartridge 110 may be a digital microfluidics (DMF)-based cartridge for performing biological analysis. DMF is a liquid-handling technology that is based upon the manipulation of microdroplets. For example, in system 100, test cartridge 110 may include a pELISA-based immunoassay panel 112, a controller 114, a communications interface 116, certain electrode drive circuitry 118 that supports any arrangements of droplet operations electrodes 120, one or more sample collection reservoirs 122, one or more reagent reservoirs 124, any arrangements of thermal control mechanisms 126, any arrangements of magnets 128, a motor drive 129, a power source 130, certain power management circuitry 132, and an EEPROM 134.

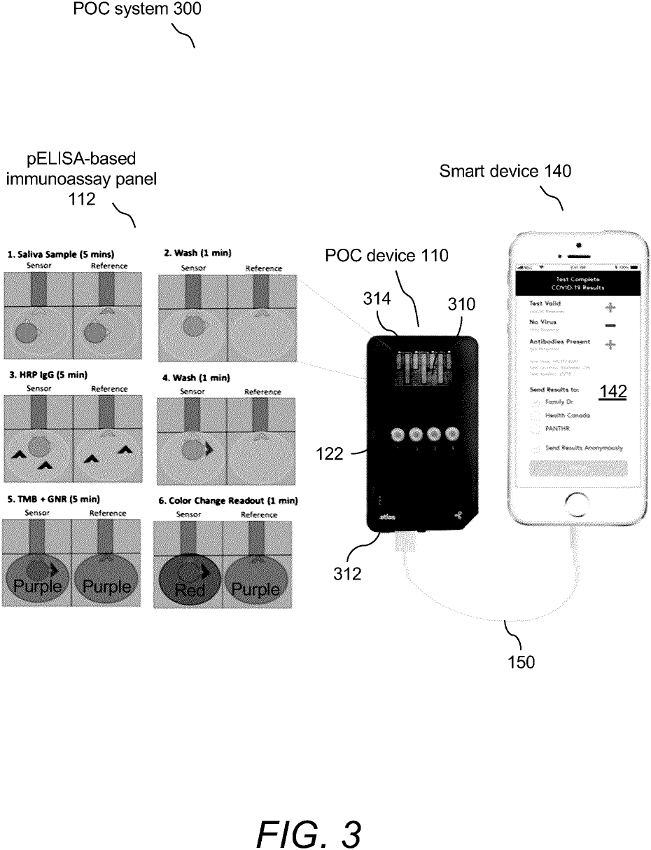

The pELISA process may be used for the detection and quantification of, for example, proteins (e.g., antibodies), viruses, bacteria, and/or any other pathogens. In test cartridge 110, the pELISA-based immunoassay panel 112 may be used to provide selective detection of multiple proteins (e.g., antibody IgG (immunoglobulin G) and antibody IgM (immunoglobulin M)) using, for example, multi-modality plasmonic sensors (see FIG. 3). For example, the pELISA process may be used to selectively detect and quantify the concentration of more than one type of protein with high specificity. Further, one or more types of particles may be used to detect multiple target proteins. More than one of the sensing modalities may be used to detect multiple target proteins with high specificity, as described, for example, in the U.S. Patent App. No. 63/104,006, entitled “Methods of plasmonic nanoparticle assisted enzyme-linked immunosorbent assay (ELISA) in a fluidics device.” Accordingly, the pELISA process may be used to provide colorimetric detection of multiple proteins in a fluidics device using multi-modality plasmonic particle sensors (i.e., particles with different sensing modalities).

Controller 114 may provide processing capabilities, such as storing, interpreting, and/or executing software instructions, as well as controlling the overall operations of test cartridge 110. The controller may be electronically coupled to any element of the test cartridge. The software instructions may comprise machine readable code stored in non-transitory memory that is accessible by the controller 114 for the execution of the instructions. Further, data storage (not shown) may be built into or provided separate from controller 114. Controller 114 may be configured and programmed to control data and/or power aspects of test cartridge 110. For example, with respect to test cartridge 110, controller 114 may control droplet manipulation by activating/deactivating droplet operations electrodes 120. Generally, controller 114 may be used for any functions of system 100. In one example, controller 114 may be an STM32F4 series microcontroller (MCU), such as the STM32F407 MCU.

Communications interface 116 may be any wired and/or wireless communication interface for electronically connecting to smart device 140 and by which information may be exchanged with smart device 140.

A wired or wireless communications link 150 may be provided for electronically coupling communications interface 116 of test cartridge 110 with smart device 140. Examples of wired communication interfaces may include, but are not limited to, USB ports, RS232 connectors, RJ45 connectors, Ethernet, and any combinations thereof.

One example of a wired communications link 150 may be the USB charging cable of smart device 140. Examples of wireless communication interfaces may include, but are not limited to, an Intranet connection, Internet, cellular networks, ISM, Bluetooth® technology, Bluetooth® Low Energy (BLE) technology, Wi-Fi, Wi-Max, IEEE 402.11 technology, ZigBee technology, Z-Wave technology, 6LoWPAN technology (i.e., IPv6 over Low Power Wireless Area Network (6LoWPAN)), ANT or ANT+ (Advanced Network Tools) technology, radio frequency (RF), Infrared Data Association (IrDA) compatible protocols, Local Area Networks (LAN), Wide Area Networks (WAN), Shared Wireless Access Protocol (SWAP), any other types of wireless networking protocols, and any combinations thereof.

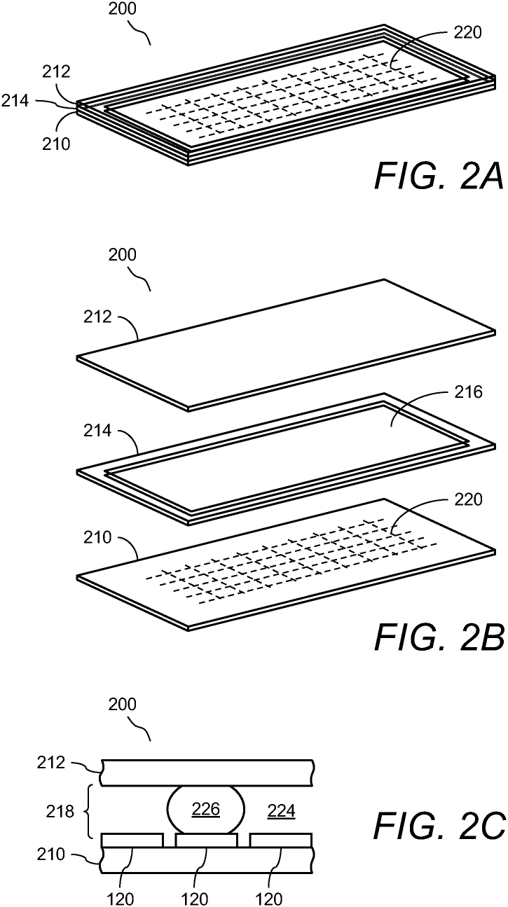

The test cartridge may include two substrates separated by a gap (i.e., a droplet operations gap) that forms a chamber in which the droplet operations are performed. Accordingly, any portion of test cartridge 110 that supports DMF may include a PCB substrate and a glass or plastic substrate separated by a gap (see FIG. 2A, FIG. 2B, FIG. 2C). Accordingly, the pELISA-based immunoassay panel 112 of test cartridge 110 may be reaction (or assay) chambers that may be supplied by any arrangements (e.g., lines, paths, arrays) of droplet operations electrodes 120 (i.e., electrowetting electrodes) and wherein the pELISA-based immunoassay panel 112 provides the detection spots of test cartridge 110.

Electrode drive circuitry 118 may be any circuitry for providing the required electrowetting voltages to the droplet operations electrodes 120. For example, electrode drive circuitry 118 may be a high voltage power supply required for DMF for creating the droplet locomotion required to run the assay. Further, the one or more sample collection reservoirs 122 and one or more reagent reservoirs 124 may supply the pELISA-based immunoassay panel 112 with the liquids to be processed.

Further, test cartridge 110 may include any other components and/or mechanisms needed to support any of the DMF operations and/or the biological analysis processes thereof. For example, thermal control mechanisms 126 may be provided for controlling the processing temperature in test cartridge 110. Thermal control mechanisms 126 may be, for example, heaters (e.g., resistive heaters), coolers, and/or any thermal sensors for controlling the heaters/coolers. Further, magnets 128 may be provided in test cartridge 110 for manipulating, for example, magnetically responsive beads. Magnets 128 may be permanent magnets and/or electromagnets. Motor drive 129 may include a DC motor and motor control circuit to provide on-demand magnet position control. Controller 114 activates the motor control circuit to move magnets 128 as required.

Power source 130 of test cartridge 110 may be, for example, a rechargeable or non-rechargeable battery. In another example, power source 130 may be power supplied by the wired communications link 150, such as the USB charging cable of smart device 140 that may supply, for example, 5V 500mA power. Power management circuitry 132 may be any circuitry for processing the power from power source 130 in a manner that is suitable for use by any active components of test cartridge 110.

EEPROM 134 may be, for example, an integrated EEPROM that may be used to store test information, such as test ID, lot ID, calibration information, usage history, and cryptographic verification. In particular, EEPROM 134 may store a standard QR code that can be decoded by mobile app 142 of smart device 140 to provide information about the test cartridge, such as: device lot number, device serial number, expiration date, date of manufacture, and assay information. In addition, the same information contained within the QR code may be printed on a label in a user readable form.

With respect to unique test cartridge identification and results sharing to a public health system, a cloud database, such as database 164 associated with networked computer 160, bookkeeps the serial numbers of test cartridges manufactured, lot number, distribution channels, manufacturing test data and other information. As test cartridges 110 are used, information about the user, test cartridge (smart device) location, results, and test cartridge status may be collected and stored. Data about the test and test cartridge is available for analysis to create reports to government agencies, and users, with the option to be submitted anonymously.

Again, smart device 140 may be used by user 105 to operate test cartridge 110 to perform the test, e.g., self-test. To that end, smart device 140 may include a mobile app 142 (or desktop application 142), an image capture device 144, an image analysis algorithm 146, and some amount of data storage 148.

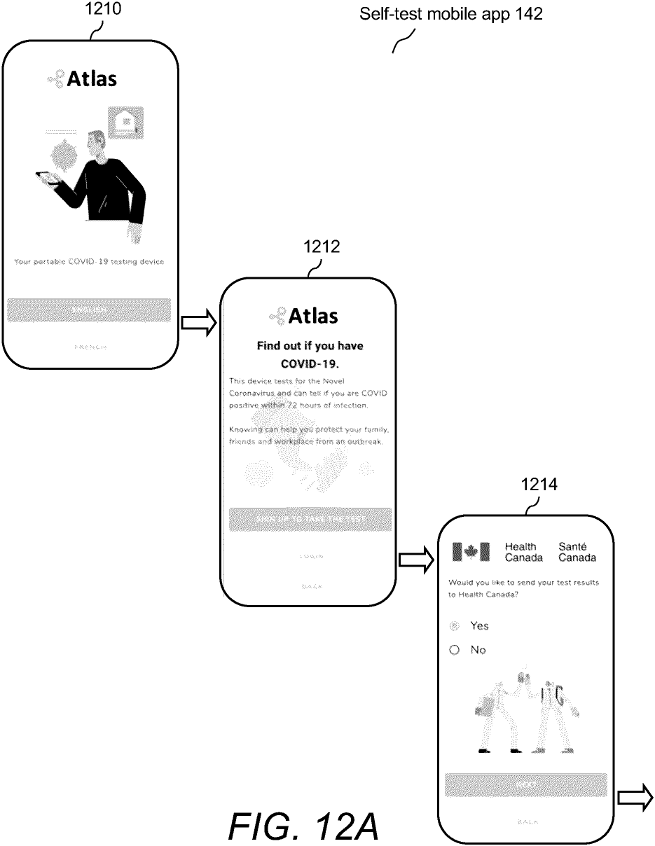

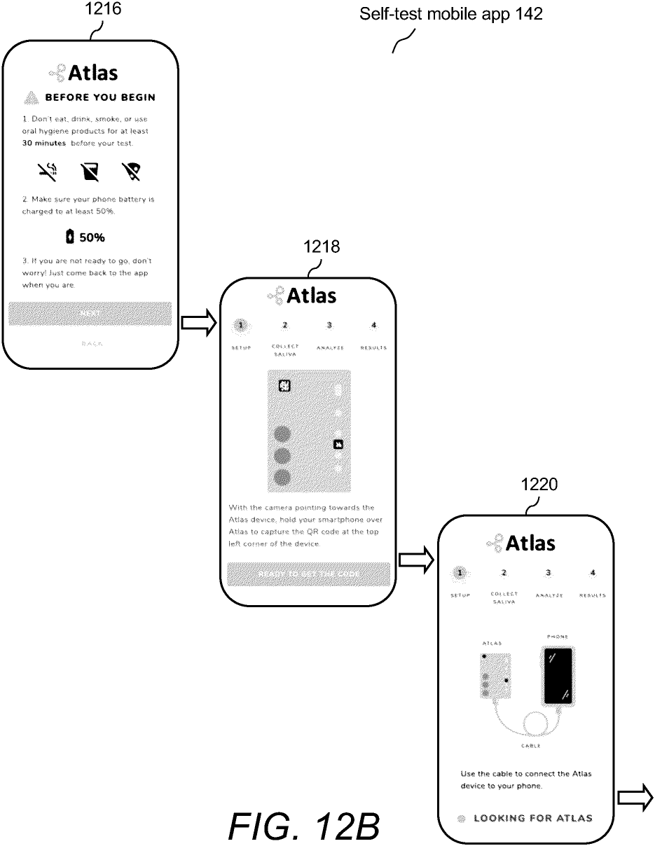

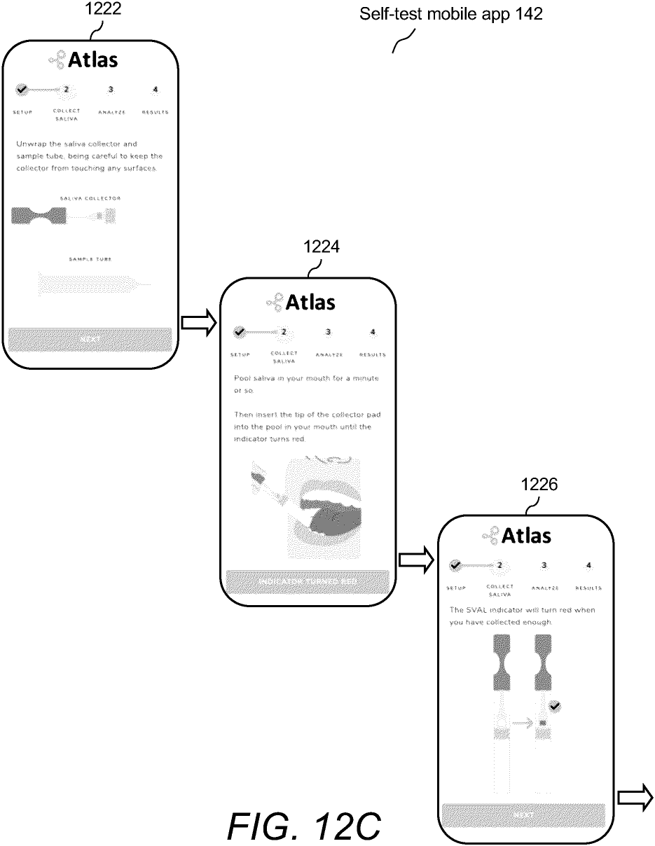

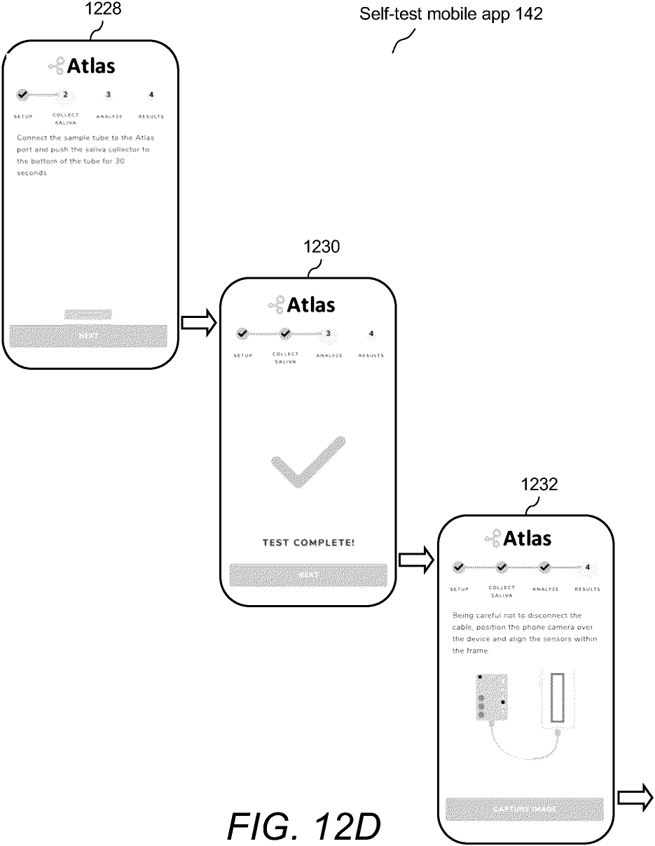

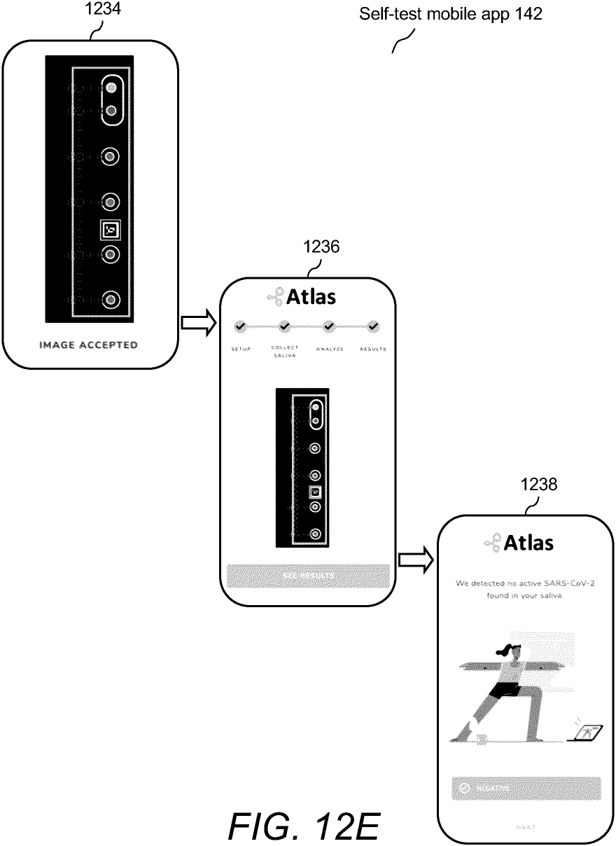

The tests of the present disclosure may be implemented using smart device app. For example, mobile app 142 provides the user interface for operating system 100 and/or test cartridge 110, as shown, for example, in FIG. 12A through FIG. 12E. Image capture device 144 may be, for example, any digital camera (e.g., standalone, or as a component of a smart device). For example, image capture device 144 may be the on-board camera of the user 105′s smart device 140 (e.g., smartphone). Image analysis algorithm 146 may be any image processing software and/or hardware for processing the digital image data from image capture device 144.

In system 100, image capture device 144 may be used to capture any colorimetric change in the sample (at pELISA-based immunoassay panel 112) due to (1) etching of plasmonic nanoparticles; (2) aggregation of plasmonic nanoparticles; (3) growth of plasmonic nanoparticles; (4) nucleation and growth of plasmonic nanoparticles by reducing metal ion precursors, and/or (5) quenching and/or unquenching the fluorescence of fluorescent probes (e.g., quantum dots). Then, image analysis algorithm 146 may be used to process any colorimetric change with respect to determining the presence or absence of the antigen of interest, such as the SARS-CoV-2 antigen.

Further, in system 100, the use of pELISA in test cartridge 110 provides increased sensitivity and lower detection limits as compared with conventional dyes (e.g., 3,3′,5,5′-tetramethylbenzidine (TMB)). Accordingly, the pELISA-based immunoassay panel 112 provides increased sensitivity of the colorimetric result such that a mobile device camera (e.g., the on-board camera of smart device 140) can be used to resolve a wide dynamic range of protein concentrations, sufficiently sensitive to enable the detection of viral antigens for detection of SARS-CoV-19 and other pathogenic infections.

Data storage 148 may be any volatile or non-volatile data storage device, such as, but not limited to, a random-access memory (RAM) device and a removable memory device (e.g., a universal serial bus (USB) flash drive). Data storage 148 may be used to store, for example, any user information, any system and/or test cartridge information (e.g., ID information), any image data from image capture device 144, any test results information, timestamp information, geolocation information, and the like.

Smart device 140 may be connected to a network. For example, smart device 140 may be in communication with a networked computer 160 via a network 162. Networked computer 160 may be, for example, any centralized server or cloud server. Network 162 may be, for example, a local area network (LAN) or wide area network (WAN) for connecting to the internet. In one example, using mobile app 142, the user 105′s test results may be transmitted from smart device 140 to networked computer 160, wherein networked computer 160 may be accessible by the user 105′s healthcare provider. A centralized database 164 of test results may be maintained at networked computer 160 to track epidemiologically relevant information, such as distribution of individuals with positive/negative test results.

Referring still to FIG. 1, the system 100 and test cartridge 110 provide a rapid, low-cost, and user-friendly mechanism that can be used at point-of-care or self-administered in the home or similar settings for testing of SARS-CoV-19 or other infectious pathogens. Self-testing may be conducted by a user in the user’s home or elsewhere. Self-testing may be conducted outside a healthcare facility.

Further, in the system 100, test cartridge 110 provides a single-use disposable device that can provide a rapid (e.g., less than 20 minutes) lab quality result from a self-collected sample which could be saliva, blood or a nasal swab. For example, test cartridge 110 may be completely self-contained and disposable, and may be operated using a standard mobile computing device for control and assay read out. The DMF-based test cartridge 110 provides precise control of the samples (e.g., saliva sample) and test reagents, and automatically performs the required assay protocols.

In the system 100, a panel of immunoassays (e.g., colorimetric ELISAs) may be automatically performed in the handheld test cartridge 110 using its DMF liquid-handling technology and then the result processed using smart device 140, which may be the user 105′s own smart device, such as a smartphone device, a tablet computing device, a laptop computing device, or the like.

In the system 100, the DMF-based test cartridge 110 may be utilized to assay microliter sized droplets of a user sample using pELISA-based immunoassay panel 112 to identify the presence of viral antigens (e.g., SARS-CoV-2) and other protein targets. Then, the test results indicated at test cartridge 110 may be immediately acquired and processed using mobile app 142 of smart device 140.

In the system 100, the integration of pELISA, DMF, and ubiquitous mobile device technology ensures an efficient diagnosis to the user and expedient communication and dissemination of these results. Leveraging smart device, such as a smartphone, technology and connectivity also enables seamless collection and analysis of data.

The pELISA-based immunoassay panel 112 can be modified or expanded to support different testing missions with potential targets including SARS-CoV-2 viral antigens, serological markers, Influenza viral antigens and other respiratory pathogen antigens. In test cartridge 110, the programmability provided by DMF enables new assay protocols to be rapidly and easily developed and deployed. This nimbleness enables it to respond to both the rapidly evolving understanding of pathogen biology and host response as well as to be quickly adapted to respond to new and emerging pathogens and pandemic threats. Such a device also enables large-scale population level screening of infections, which is critical for the containment of pandemic infections.

In one embodiment, test cartridge 110 uses a single saliva sample as an input to perform a pELISA assay to determine SARS-Cov-2 infection status by direct viral antigen detection. The results can be read by the naked eye as a color change but also, quantitatively, by using the imbedded or on-board camera module (e.g., image capture device 144) of a mobile computing device (e.g., smart device 140) to improve accuracy and enable digital recording and transmission. Further, test cartridge 110 may be expanded to test for multiple infection markers. For example, this could include serological markers like IgG, IgM, or other viruses like Influenza A or B, or RSV to provide a differential diagnosis.

Although many different sample types are compatible with test cartridge 110, saliva is preferred because it can be easily self-collected at home thereby avoiding contact with others and minimizing sample collection errors. Several recent reports indicate the appropriateness of saliva samples for testing of SARS-CoV-2 virus and COVID-19 infection. Studies have also shown that IgG can be measured in saliva with good correlation to serum levels, enabling the determination of previous infections.

The use of DMF in test cartridge 110 allows automation of all steps of the ELISA which are traditionally performed by a trained lab technician. DMF enables precise manipulation of small quantities of liquid (i.e., samples, reagents, buffers) by using the principle of electrowetting to directly manipulate liquid droplets and perform basic operations such as aliquoting, mixing, splitting, and incubating, which are directly analogous to traditional benchtop methods. Compared to lateral flow immunoassays (LFIAs) which lack any active fluid control, DMF allows far more complex assays to be performed and minimizes the potential for human error. Active mixing using DMF also enables faster reactions which substantially reduces the ELISA time to result.

A traditional DMF system consists of a benchtop instrument containing the electronics and sensors and a separate disposable cartridge which performs the droplet operations. The DMF cartridge typically consists of a PCB carrying an array of electrodes that is covered by a plastic top-plate which provides access for loading samples and reagents, as shown, for example, in FIG. 2A, FIG. 2B, and FIG. 2C. The electrowetting-driven droplet operations occur in a thin gap between the two parts.

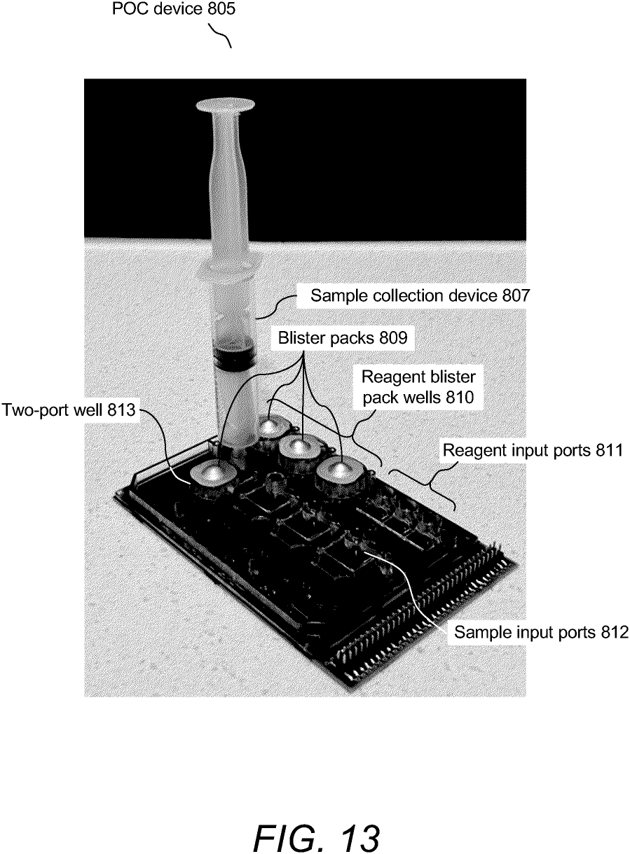

A main benefit of test cartridge 110 of the system 100 is that it may include a DMF cartridge portion (e.g., PCB bottom substrate and plastic top substrate separated by a gap) and a control electronics PCB integrated together in one assembly. More details of an example of an integrated test cartridge 110 are shown and described hereinbelow with reference to FIG. 13 through FIG. 26.

Another benefit of test cartridge 110 of the system 100 is that a mobile computing device may be used for control, detection, analysis, communications, and power, removing the need for any other equipment, making the entire device a low-cost disposable test. In this regard, the PCB of test cartridge 110 may comprise a standard or proprietary communication port (e.g., a USB charging cable) of the mobile computing device (e.g., smart device 140) to allow for electrical interface between test cartridge 110 and the mobile computing device. Further still, wireless technologies and protocols may be leveraged to facilitate communication between test cartridge 110 and the mobile computing device including, without limitation, IEEE 802.11 (Wi-Fi), Bluetooth, Zigbee, NFC, RFID, etc.

System 100 may combine the functionality of the instrument and disposable cartridge into a single disposable device (e.g., test cartridge 110) enabling equipment-free operation.

In the system 100, for instrument-free readout, pELISA enables a direct visual or smart device, such as a smartphone-assisted readout. By comparison, traditional ELISAs use horseradish peroxidase (HRP) enzyme to convert 3,3′,5,5′-Tetramethylbenzidine (TMB) into TMB+ and/or TMB2+, which turns the solution various shades of yellow depending on the concentration of target antigen. However, this is challenging to distinguish without dedicated instrumentation. Through the addition of gold nanostructures, the color change can be greatly amplified due, for example, to etching of the gold surface, which is proportional to the TMB+ and/or TMB2+ concentration. This increases the assay sensitivity by at least 10X and makes instrument-free read out possible.

Test cartridge 110 of system 100 is well positioned for rapid, high volume manufacturing. The DMF portion of test cartridge 110 may be made from standard PCB and injection molded plastic parts. By way of example, FIG. 2A, FIG. 2B, and FIG. 2C show a perspective view, an exploded view, and a cross-sectional view of an example of a basic DMF structure 200 for forming the DMF portion of test cartridge 110.

In this example, DMF structure 200 may include a bottom substrate 210 and a top substrate 212 with a spacer or gasket 214 arranged therebetween. A space or opening 216 in spacer or gasket 214 provides a droplet operations gap 218 between bottom substrate 210 and a top substrate 212. Bottom substrate 210 may be, for example, a PCB. Top substrate 212 may be, for example, a plastic or glass substrate. An electrode configuration 220 may be provided on bottom substrate 210, the PCB. As shown in FIG. 2C, electrode configuration 220 may include an arrangement of droplet operations electrodes 120. Further, droplet operations gap 218 may be filled with a filler fluid 224. Filler fluid 224 may, for example, be or include a low-viscosity oil, such as silicone oil or hexadecane filler fluid. Further, FIG. 2C shows an example of a droplet 226 atop droplet operations electrodes 120.

FIG. 3 is a system 300, which is an implementation of the system 100 and test cartridge 110 shown in FIG. 1. In this example, test cartridge 110 of system 300 may include a DMF device 310 installed in a housing 312. DMF device 310 and housing 312 are sized such that test cartridge 110 may be a handheld device. The pELISA-based immunoassay panel 112, controller 114, communications interface 116, electrode drive circuitry 118, droplet operations electrodes 120, sample collection reservoirs 122, reagent reservoirs 124, thermal control mechanisms 126, magnets 128, motor drive 129, power source 130, power management circuitry 132, and EEPROM 134 described in FIG. 1, while not necessarily visible in FIG. 3, are installed on or with respect to DMF device 310. FIG. 3 shows, for example, four sample collection reservoirs 122 in housing 312.

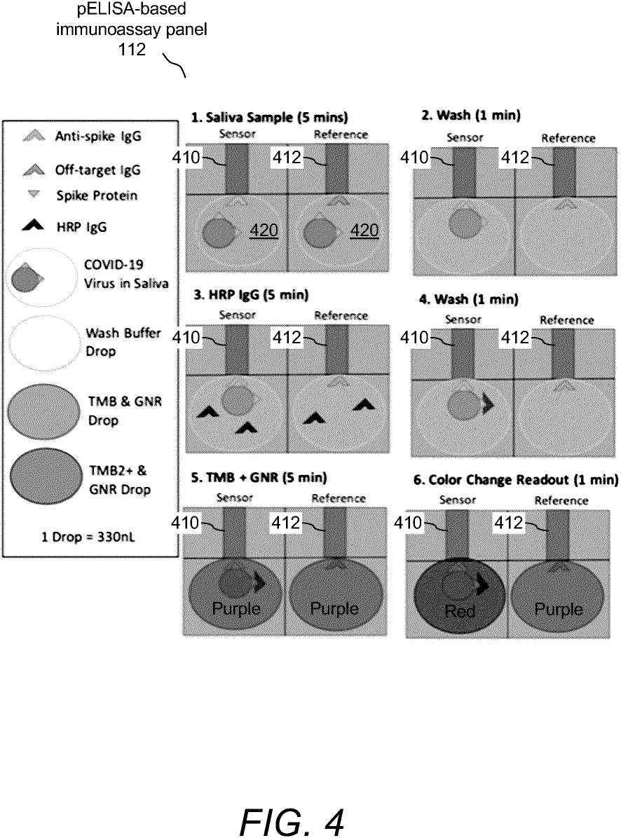

A viewing window 314 is provided in housing 312 for viewing the portion of DMF device 310 that includes the pELISA-based immunoassay panel 112. By way of example, FIG. 4 shows a schematic diagram of an example of a pELISA assay performed at the pELISA-based immunoassay panel of the system 100 and test cartridge 110. Also note that the same steps are applicable to any antigen of interest.

In the pELISA assay shown in FIG. 4, there are six (6) steps to the assay that may be completed in, for example, about 18 minutes, completely automatically by DMF in test cartridge 110. There are separate detection spots, for example, a viral antigen detection spot 410, a second antigen (e.g., IgG) detection spot 412, a positive control detection spot (not shown), and a negative control detection spot (not shown). Further, a background color reference detection spot may be provided for image recognition. The viral antigen detection spot 410 uses an immobilized anti-spike antibody to capture the intact virus. The positive control detection spot (not shown) uses protein A to capture generic salivary IgG, and the background reference detection spot (not shown) is a blocked surface. After introducing the saliva sample 420 to each sensor (e.g., step 1 followed by a wash step 2), HRP labeled secondary antibodies are introduced which bind to the sensors if the target was present in the saliva sample (e.g., step 3 followed by a wash step 4). The HRP substrate TMB and gold nanorods (GNRs) are introduced, which will change in color from purple to red based on the amount of HRP present and indicating a positive result (e.g., step 5 and step 6). By having on-board positive and negative controls, the user/smart device, such as a smartphone, read-out is much more accurate. In this example, the affinity reagents are dried on the DMF device 310, so the user only needs to add the provided buffer and TMB+GNR solution.

### Test Methods and Systems

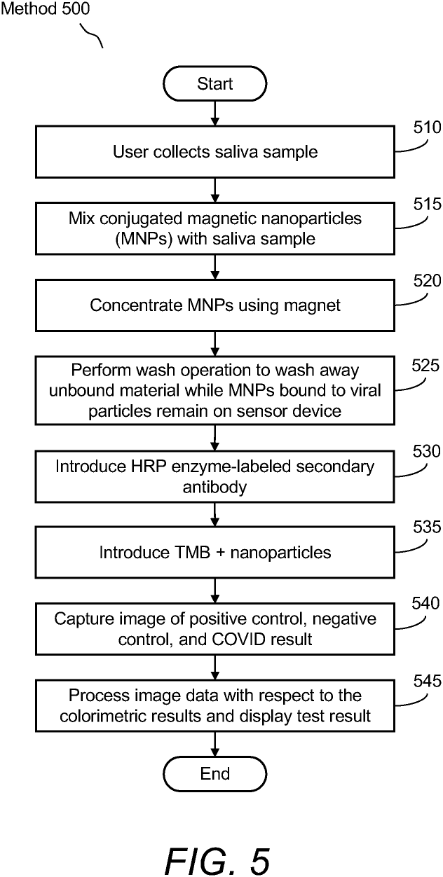

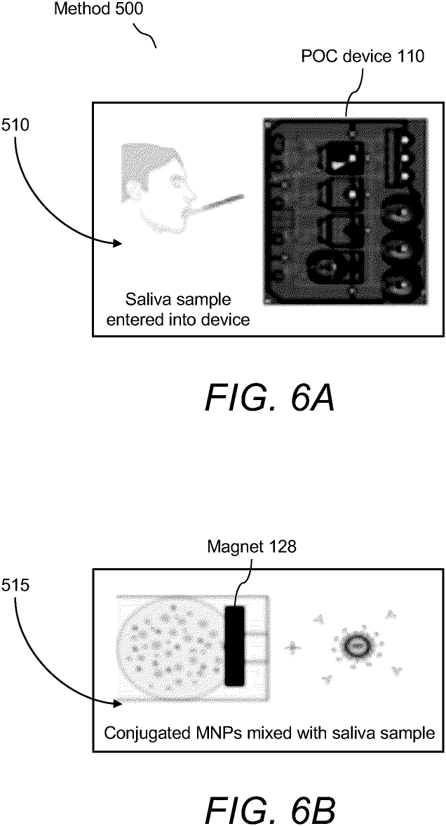

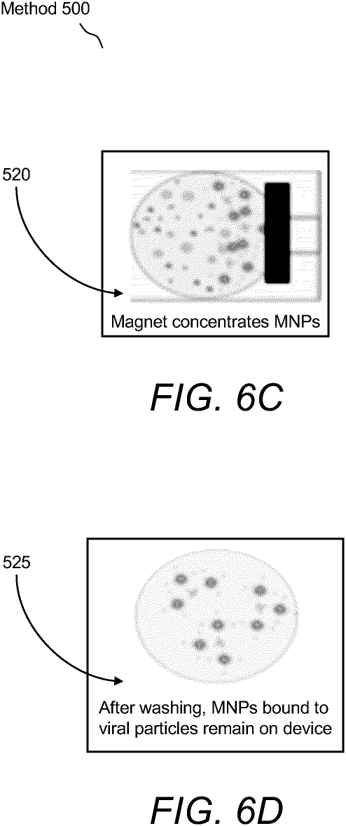

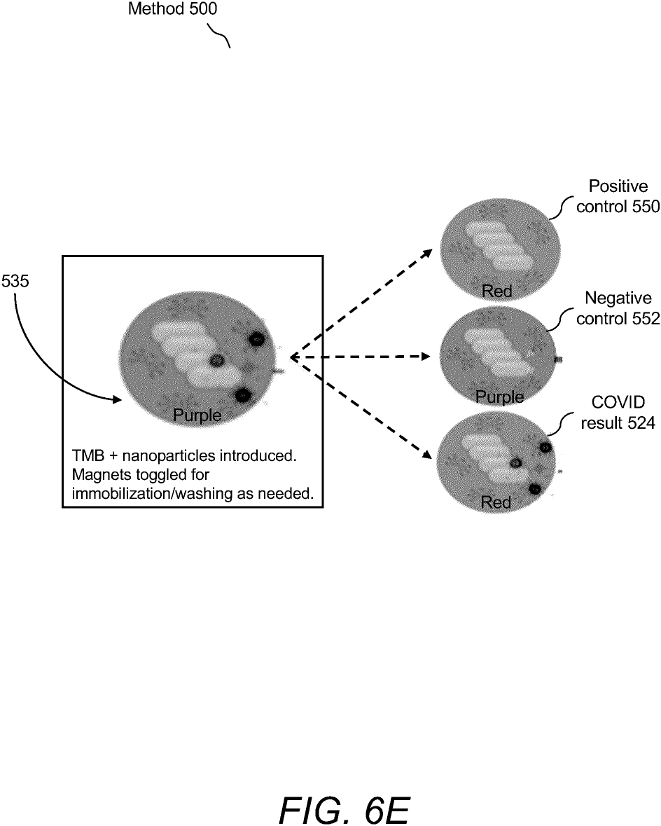

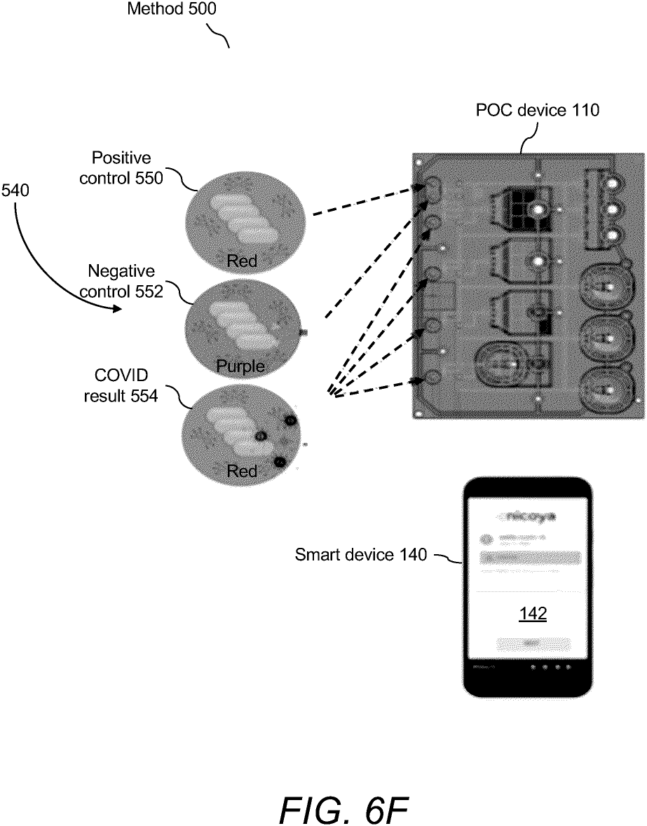

FIG. 5 is a flow diagram of an example of a method 500 of performing an pELISA assay on the system 100 and test cartridge 110. Further, FIG. 6A through FIG. 6F shows pictorially an example of certain steps of method 500.

The system 100, test cartridge 110, and method 500 provide a color-based detection means with respect to determining the presence or absence of a virus (e.g., SARS-CoV-2). Method 500 may include, but is not limited to, the following steps as well as additional unspecified steps.

At a step 510 and pictured in FIG. 6A, user 105 collects a sample of his/her saliva. Then, the saliva sample is inserted into test cartridge 110. For example, user 105 collects his/her saliva sample using a swab. Then, the swab with the saliva sample thereon is inserted into one of the sample collection reservoirs 122 of test cartridge 110.

At a step 515 and pictured in FIG. 6B, conjugated magnetic nanoparticles (i.e., MNPs with viral anti-spike protein antibodies bound thereon) are mixed with the saliva sample. For example, at the pELISA-based immunoassay panel 112 of test cartridge 110 and using droplet operations, conjugated MNPs (e.g., magnetically responsive beads) are mixed with the saliva sample.

At a step 520 and pictured in FIG. 6C, the MNPs are concentrated via a magnet. For example, at the pELISA-based immunoassay panel 112 of test cartridge 110 and using droplet operations, the MNPs are concentrated via a magnet 128.

At a step 525 and pictured in FIG. 6D, a wash operation is performed to wash away unbound material. For example, at the pELISA-based immunoassay panel 112 of test cartridge 110 and using droplet operations, a wash operation is performed to wash away any unbound material. After washing, MNPs bound to viral particles remain on the device.

At a step 530, an HRP enzyme-labeled secondary antibody is introduced. For example, at the pELISA-based immunoassay panel 112 of test cartridge 110 and using droplet operations, an HRP enzyme-labeled secondary antibody is introduced and binds to the virus-laden MNPs. Then, magnets 128 may be toggled for immobilization/washing as needed.

At a step 535 and pictured in FIG. 6E, TMB + nanoparticles are introduced. For example, at the pELISA-based immunoassay panel 112 of test cartridge 110 and using droplet operations, TMB + nanoparticles (e.g., gold nanourchins) are introduced. Then, magnets 128 may be toggled for immobilization/washing as needed. FIG. 6E also shows an example of the detection spots of the pELISA-based immunoassay panel 112 for a positive control 550, a negative control 552, and a COVID result 554.

At a step 540 and pictured in FIG. 6F, an image is captured of the detection spots of test cartridge 110. For example, under the guidance of mobile app 142 of smart device 140, user 105 captures an image of positive control 550, negative control 552, and COVID result 554 via the image capture device 144 of smart device 140. In one example, FIG. 6F shows that test cartridge 110 may include one positive control 550 detection spot, one negative control 552 detection spot, and four COVID result 554 detection spots.

At a step 545 and pictured in FIG. 6F, the image data is processed, and the test result is displayed to the user. For example, image analysis algorithm 146 of smart device 140 is used to process the image data from image capture device 144 with respect to the colorimetric results. Then, mobile app 142 is used to display the test result to user 105.

In some embodiments, the pELISA assay uses magnetically responsive beads for initial virus concentration and subsequent assay processing steps. The magnetically responsive beads are bound to the virus though a high affinity anti-spike protein antibody, which is then concentrated into a single 330 nl droplet. The HRP enzyme labeled secondary reporter antibody is then introduced and binds to the virus-laden magnetically responsive beads, and upon addition of the substrate (e.g., TMB), generates a large concentration of oxidized substrate. This oxidized substrate is combined with gold nanourchins, which etches the nanourchins, causing a visual color change. Using smart device 140, the easy-to-use mobile app 142 enables image capture device 144 and image analysis algorithm 146 to interpret the colorimetric results from the assay.

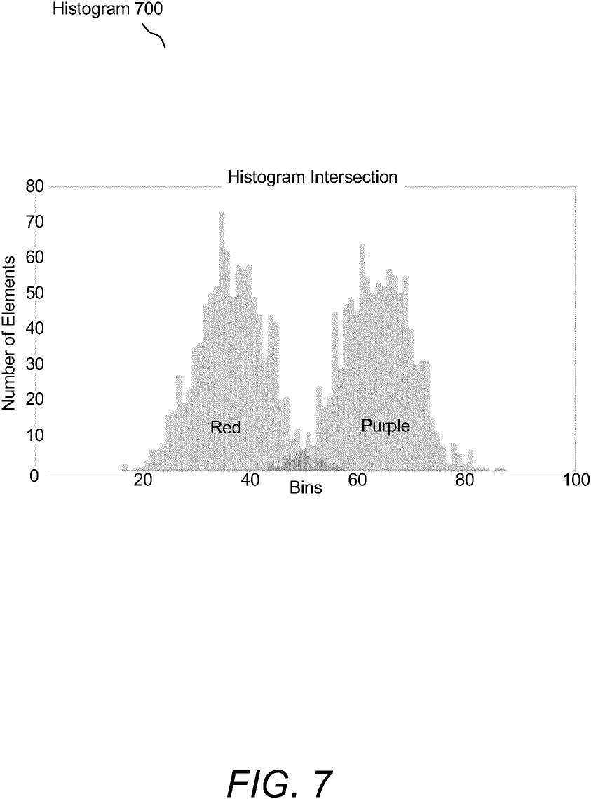

With respect to the smart device, such as a smartphone, image analysis for result readout, FIG. 7 shows an example of an image analysis histogram 700 generated using image analysis algorithm 146 of smart device 140. To account for any user variation, mobile app 142 and image analysis algorithm 146 of smart device 140 are provided to automate image analysis for results interpretation. Mobile app 142 guides user 105 to align and capture a photo with controls and user samples included. An ArUco fiducial marker is built-in for the app to guide user 105 for positional/distance alignment as well as for acceptable pitch, yaw, and roll position. A matrix of 5x5 pixels is saved from each of the reference and user samples and are converted from RGB to HSV (Hue, Saturation, Value) space. Normalized histograms of the samples and references are compared against threshold values to determine Positive, Negative or indeterminate results, which are sent to user 105 and to database 164. A sample image analysis histogram 700 is shown in FIG. 7. A difference algorithm is used to compare the histogram and measure the difference to determine the result. The more overlap in bins (X values) & Y values, the more similar they are. Other algorithms (not shown) may be used to leverage the cloud, and eliminate dependencies based on the computing power, memory availability, and operating system.

ELISA reagents are commercially available as are the antibodies and antigens required for COVID-19 testing and many other pathogens. All components required for the electronics of test cartridge 110 are currently available as commoditized parts.

The required drive electronics may be fully or in part integrated into test cartridge 110. For example, the functionality for high-voltage generation, multiplexing, waveform generation, and droplet feedback control may be integrated into test cartridge 110. An integrated EEPROM can be used to store all required test information, such as test ID, lot ID, calibration information, usage history, and cryptographic verification. Cost of goods sold (COGS) for test cartridge 110 is estimated to be low, supporting the use of test cartridge 110 as a single-use and disposable device which enables testing in the home or similar locations and disposal without the need to decontaminate a fixed instrument between runs. Again, test cartridge 110 may be controlled using a standard smart device 140 using a dedicated application (e.g., mobile app 142) to control and monitor the assay and analyze and communicate the results.

### Test Cartridge Design and Interface With Smart Device

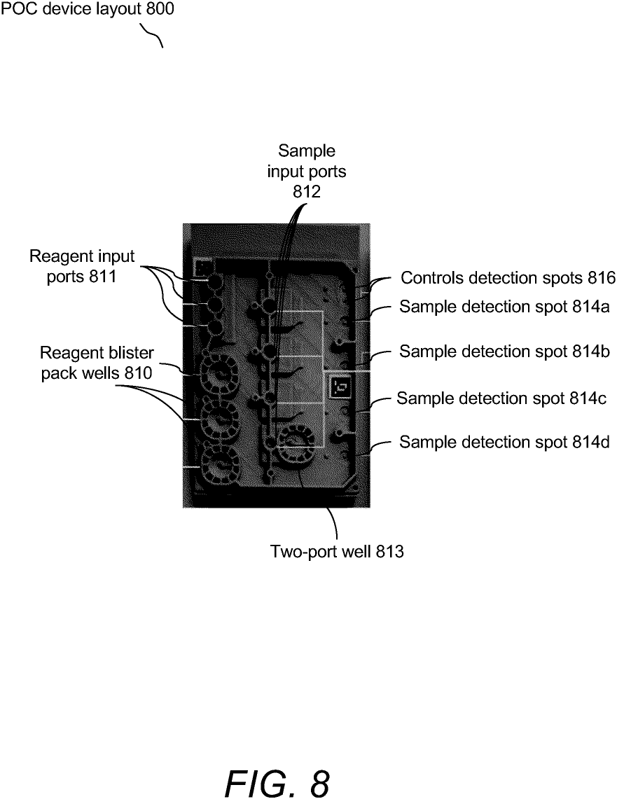

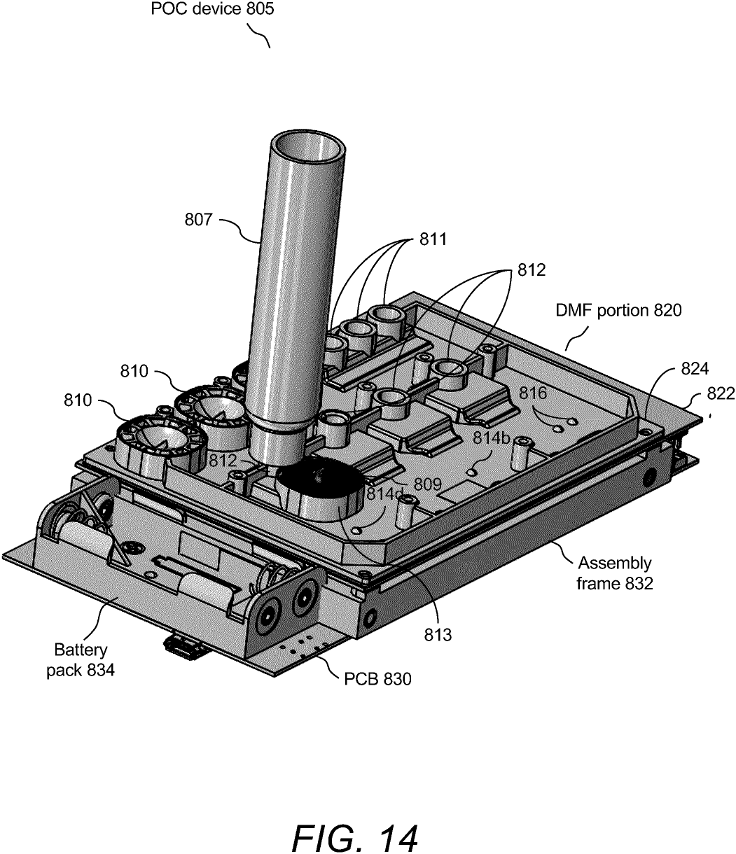

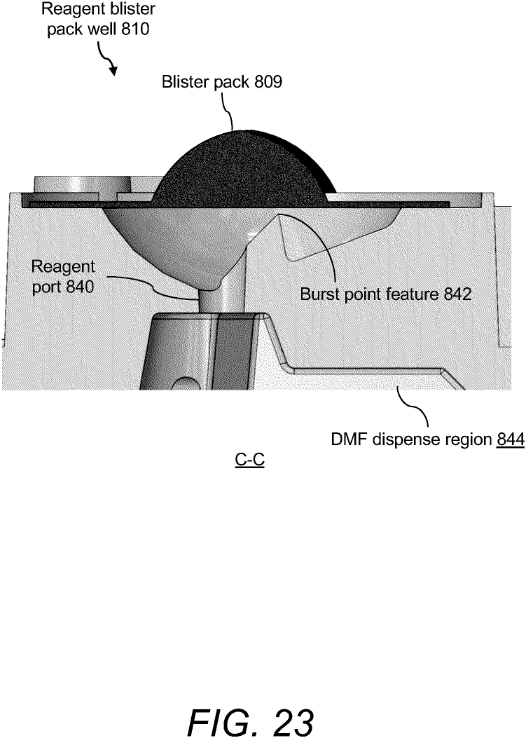

FIG. 8 is a photo of an example of a test cartridge layout 800 that may be representative of the test cartridge 110. In this example, test cartridge layout 800 may include three reagent blister pack wells 810, three reagent input ports 811, four sample input ports 812, four sample detection spots 814 (e.g., 814a, 814b, 814c, 814d), and two controls detection spots 816 (e.g., one positive and one negative control). Further, one of the sample input ports 812 may be one input of a two-port well 813. The three reagent blister pack wells 810 as well as the two-port well 813 may be sized and shaped to hold prepackaged reagents in the form of blister packs (see FIG. 13 and FIG. 14). The second input to the two-port well 813 may be a blister pack (see FIG. 13 and FIG. 14). In one example, each of the three reagent blister pack wells 810 as well as the two-port well 813 may hold about 40 µl and each of the wells supplied by sample input ports 812 may hold up to about 500 µl. Test cartridge layout 800 may also include a mechanical crush-plate or other bursting mechanism in relation to the blister packs (see FIG. 23 and FIG. 24).

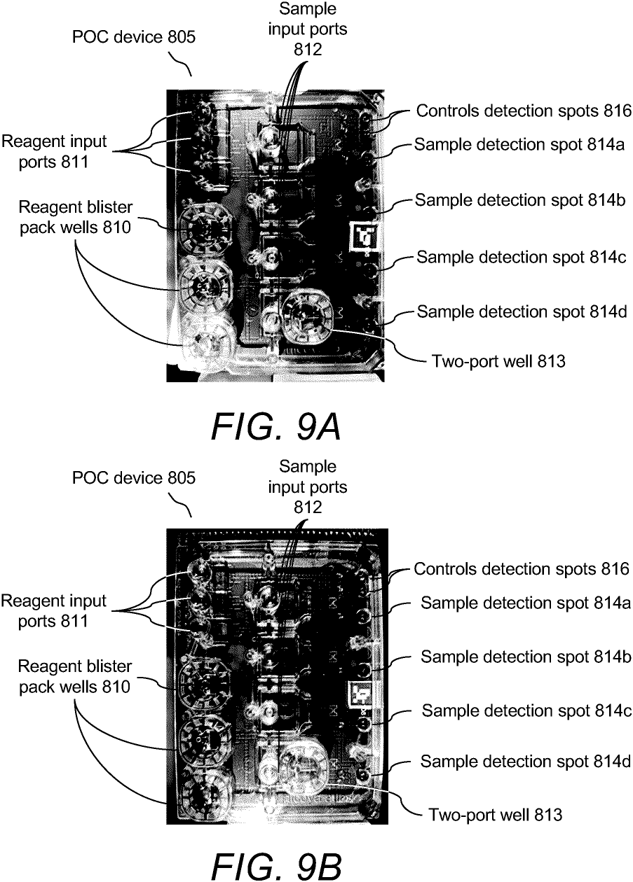

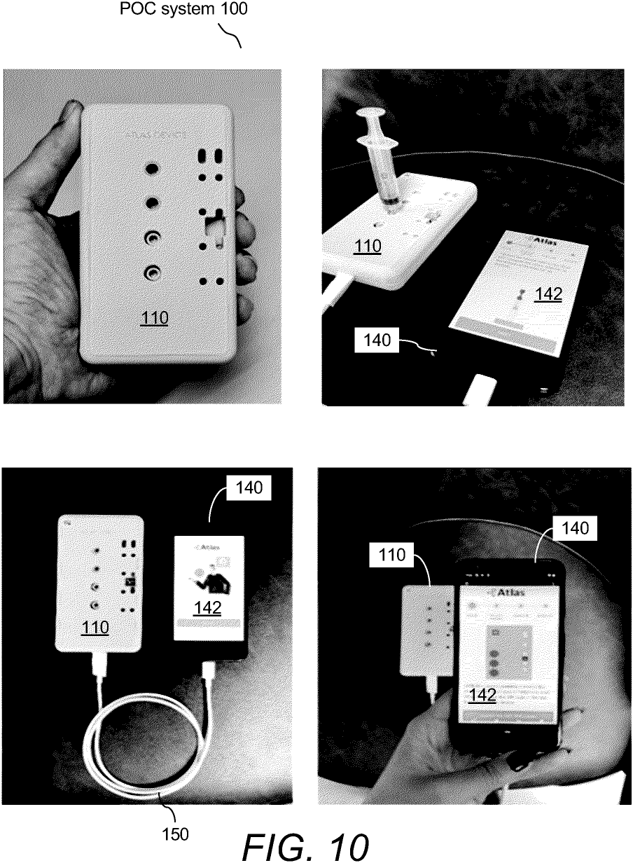

FIG. 9A and FIG. 9B is photos of a DMF device 805, which may be an example of the test cartridge 110 shown in FIG. 1. Further, FIG. 9A and FIG. 9B show the DMF-portion of DMF device 805. For example, FIG. 9A shows the detection of HRP labeled magnetic beads. The detection spots 814a, 814b, 814c, 814d, 816 are located in the red rectangle. FIG. 9B shows the detection of 10 nM spike protein captured with R001-magnetic beads and read out with pELISA. Again, the detection spots 814a, 814b, 814c, 814d, 816 are located in the red rectangle. More details of DMF device 805 are shown and described hereinbelow with reference to FIG. 13 through FIG. 26.

FIG. 10 is another implementation of the system 100 and test cartridge 110 suitable for performing test, such as a self-test. In this example, test cartridge 110 may be a somewhat more miniaturized device compared with the test cartridge 110 shown, for example, in FIG. 3. In this example, test cartridge 110 may include drive electronics and DMF electrodes integrated into one PCB. Further, test cartridge 110 may include a horizontal sliding magnet to help achieve a compact design. In this example, test cartridge 110 can accommodate four (4) patient samples at a time and also has the flexibility to be battery operated aside from being USB-powered.

### Test Workflow

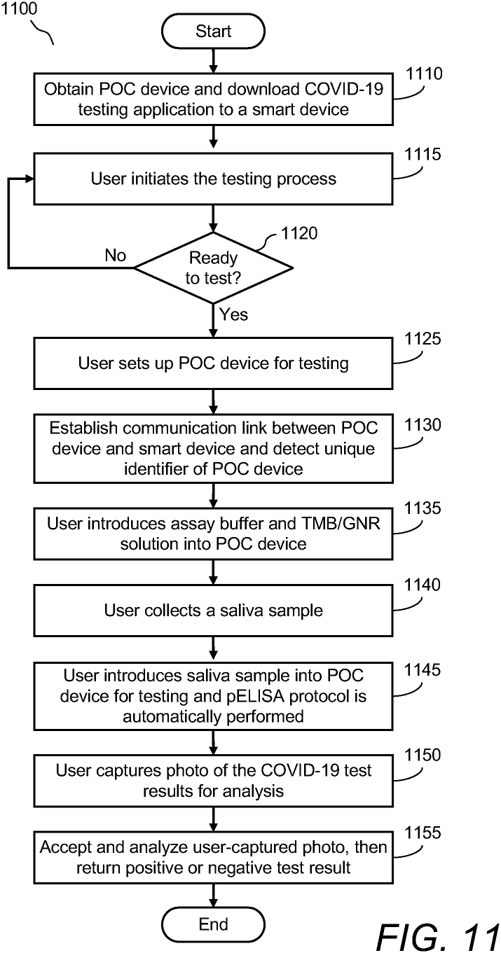

FIG. 11 is a flow diagram of an example of a user workflow 1100 for testing, e.g., self-testing, for SARS-Cov-2 viral infection, which is the causative agent of COVID-19, using the system 100 and test cartridge 110. Further, FIG. 12A through FIG. 12E show screenshots of an example of a user interface of mobile app 142 of smart device 140. FIG. 12A through FIG. 12E show pictorially an example of certain steps of user workflow 1100. User 105 may be guided through the entire user workflow 1100 by instructions provided on mobile app 142 of the user 105′s smart device 140.

In order to make test cartridge 110 easy enough for a non-specialist to use reliably, the overall workflow must be as simple and error-proof as possible. While there are many possible variations, an example user workflow 1100 may include, but is not limited to, the following steps as well as additional unspecified steps.

At a step 1110, a test cartridge is obtained, and a COVID-19 testing application is downloaded to a smart device. For example, a user 105 obtains test cartridge 110 and reads the instructions on the packaging of test cartridge 110 on how to download and open mobile app 142 on his/her smart device 140. In one example, smart device 140 is the user’s smart device, such as a smartphone. This step is also shown pictorially in screenshot 1210 of FIG. 12A.

At a step 1115, the user initiates the testing process. For example, guided by mobile app 142, user 105 selects a language (e.g., English or French) and logins into mobile app 142 to take the COVID-19 test. Guided by mobile app 142, user 105 also chooses whether to share their testing data with a surveillance database (e.g., his/her national and/or regional public health department (e.g., Health Canada)). This step is also shown pictorially in screenshots 1212 and 1214 of FIG. 12A.

At a decision step 1120, the user determines whether he/she is ready to proceed with the testing process. For example, guided by mobile app 142, user 105 decides whether he/she complies with pre-testing conditions and whether his/her smart device 140 (e.g., his/her smart device, such as a smartphone) has sufficient battery charge for performing the test. If yes, the user is ready to take the test, then method 1100 proceeds to a step 1125. If no, the user is not ready to take the test, then method 1100 returns to step 1115. This step is also shown pictorially in screenshot 1216 of FIG. 12B.

At a step 1125, the user sets up the test cartridge for testing. For example, guided by mobile app 142, user 105 uses the camera on his/her smart device 140 (e.g., his/her smart device, such as a smartphone,) to capture the QR code at the top left-hand corner of test cartridge 110. Mobile app 142 reads the QR code and stores the information using EEPROM 134. Mobile app 142 also collects and stores other required test information such as device lot number, device serial number, expiration date, date of manufacture, and assay information. This step is also shown pictorially in screenshot 1218 of FIG. 12B.

At a step 1130, a communication link is established between the test cartridge and the smart device and the unique identifier of the test cartridge is detected. For example, user 105 connects test cartridge 110 to his/her smart device 140 using, for example, the USB charging cable of the smart device 140 (e.g., the USB charging cable of the user’s smart device, such as a smartphone,). In this step, smart device 140 detects a unique identifier on EEPROM 134. At the completion of the setup process, mobile app 142 displays, for example, a checkmark in a “setup” box of a test progress timeline display indicating to the user that this step in the testing process has been completed. This step is also shown pictorially in screenshot 1220 of FIG. 12B.

At a step 1135, the user introduces assay buffer and TMB/GNR solution into the test cartridge. For example, guided by mobile app 142, user 105 inserts 2-3 drops of the assay buffer and TMB/GNR solution into test cartridge 110 via reagent reservoirs 124. In another example, pre-packaged reagents in the form of blister packs may be provided on test cartridge 110. In this example, user 105 may be prompted to engage any corresponding mechanisms to puncture these blister packs. For example, test cartridge 110 may include a mechanical crush-plate for puncturing or bursting these blister packs.

At a step 1140, the user collects a saliva sample. For example, guided by mobile app 142, user 105 takes a syringe and collector tube (i.e., saliva collector device) out of the test cartridge packaging, as shown pictorially in screenshot 1222 of FIG. 12C. Then, guided by mobile app 142, user 105 places the syringe and collector tube under their tongue for about 1 minute, as shown pictorially in screenshot 1224 of FIG. 12C. An indicator on the syringe may turn red when enough saliva has been collected, as shown pictorially in screenshot 1226 of FIG. 12C.

At a step 1145, the user introduces their saliva sample into the test cartridge for testing. For example, guided by mobile app 142, user 105 connects the syringe and collector tube (i.e., saliva collector device) to one of sample reservoirs 122 of test cartridge 110, as shown for example in FIG. 10, FIG. 13, and FIG. 14. Then, user 105 presses a plunger into the syringe and collector tube to fully compress and deliver the saliva sample into test cartridge 110 for testing. Upon introduction of the saliva sample into test cartridge 110, mobile app 142 displays, for example, a checkmark in a “collect saliva” box of the test progress timeline display indicating to the user that this step in the testing process has been completed, as shown pictorially in screenshot 1228 of FIG. 12D. After successful loading of the user’s saliva sample, the user initiates the test (i.e., the pELISA detection process) using mobile app 142. Then, upon completion of the pELISA detection process, a “Test complete” may be indicated to user 105, as shown pictorially in screenshot 1230 of FIG. 12D. Mobile app 142 also records and stores, for example, the time, date, and geolocation of the test.

At a step 1150, the user captures a photo of the COVID-19 test results for analysis. For example, guided by mobile app 142, user 105 is instructed to align the camera in his/her smart device 140 (e.g., his/her smart device, such as a smartphone,) over test cartridge 110 and capture a photo of the test results (e.g., the pELISA-based immunoassay panel 112) for the positive and negative controls and user test sample within a marked analysis window. This step is also shown pictorially as screenshots in screenshot 1232 of FIG. 12D.

At a step 1155, the user-captured photo is accepted and analyzed, and a positive or negative test result is returned. For example, mobile app 142 accepts and processes the captured test images and analyzes the color readouts from the positive and negative controls and user saliva sample to determine the presence or absence of SARS-CoV-2 antigen. At the completion of the image analysis process, mobile app 142 displays, for example, a negative test result message “Negative, we detected no SARS-CoV-2 in your saliva sample” or a positive test result message “Positive, we detected SARS-CoV-2 in your saliva sample”. If the user selected in step 1115 to share his/her test data with a surveillance database, the test results are sent by mobile app 142 to the surveillance database (e.g., database 164) via network 162. This step is also shown pictorially in screenshots 1234, 1236, and 1238 of FIG. 12E.

Referring still to FIG. 11, in another embodiment of user workflow 1100, at step 1145 the test may be initiated automatically using, for example, (1) a sensor or button in test cartridge 110 triggered by the syringe during sample introduction, or (2) capacitive detection of fluid being added to test cartridge 110.

Referring still to FIG. 11, in another embodiment of user workflow 1100, step 1135 may be executed after steps 1140 and 1145. In this scenario, the test may be initiated automatically when the crush-plate that bursts the blisters in step 1135 is actuated, also activating a button or sensor.

Referring still to FIG. 11, in another embodiment of user workflow 1100, steps 1135 and 1145 may be performed out of order and the test begins automatically when both steps are executed as above.

Referring now again to FIG. 1 through FIG. 12E, below are listed a set of desirable requirements for an easy-to-use POC diagnostic for SARS-CoV-2 detection that a device, such as test cartridge 110, may likely meet or exceed:

Detect SARS-CoV-2 (the causative agent of COVID-19) within the first 72 hours of symptoms with an accuracy similar to molecular RNA detection.
Results available 20 minutes (at most) from sample collection to diagnosis
Sample input not a nasopharyngeal swab
Single use device
Usable by a lay person with no technical training
Interpretation of the result should be easy for a lay person (e.g., negative or positive)
Each test cartridge possessing a unique identifier (i.e., serial code, QR or barcode).

The use of a smart device 140, e.g., smartphone, to control test cartridge 110 offloads much of the cost and complexity from the device supporting its use as single-use and disposable.

In operation, mobile app 142 may instruct user 105 to take a photo of the assay result region (e.g., the pELISA-based immunoassay panel 112) once the test is complete, with markings on the outer case used to line up the camera to the correct location. Once the photo is taken, image processing is performed to determine whether each test is positive or negative. The pELISA technology ensures that the substantial color change is able to be analyzed by the camera to easily distinguish between a positive and negative result even in varying lighting conditions, removing the ambiguity of a visual read out. This is ensured by having the positive control, negative control, and/or background color reference tests that will also produce reference positive and negative results.

Reference marks may be included on test cartridge 110 to facilitate image processing, such as marks used for alignment of the image to facilitate determination of the assay result portion of the image, and/or marks to trigger the phone when alignment between image capture device 144 of smart device 140 and test cartridge 110 is sufficient to trigger image capture.

Multiple images from slightly different angles may be captured for the same assay. For smart devices with multiple cameras this could be done using those multiple cameras, or multiple images from slightly different angles may be captured manually or may be chosen from a video sequence. This will help the algorithm more clearly distinguish stray light and angular lighting effects.

Many mobile devices have a reverse-facing camera as well, popularly called a selfie camera. This camera can be used to simultaneously capture a reverse image while the assay image is being captured. The Fresnel reflection will primarily be of the elements captured in this reverse image, and thus the reverse image may be used to better remove effects of the Fresnel reflection. Furthermore, the ambient light can also be independently estimated using the reverse image.

Apart from standard image processing techniques such as contrast stretching and noise reduction (not necessary if a full-fledged statistical estimation algorithm is being implemented), machine learning algorithms may be used as well. Machine learning algorithms may be especially suited to estimate the confidence (or variance) in the answer produced by the primary statistical estimation algorithm and may learn to utilize secondary cues present in images such as the presence of fluorescent light, shadows, etc., especially from the reverse image.

Many mobile cameras now allow control over exposure, gain and even focus. For example, Android’s camera2 API allows such control, though the implementation will vary per Android device. Using such an API, capture parameters can be controlled in a beneficial fashion to capture the most useful image. In this way, capturing an image that is not overexposed, nor too dark can be achieved.

If an image is badly focused, this will cause colors to bleed into each other, thus hampering the accuracy of the algorithm. Focus quality can be assessed algorithmically. Image correction may be accomplished algorithmically. The user may be prompted via the smart device to retake the image.

In indoor lighting conditions, relatively long exposures may be needed, thus creating the possibility that a slightly trembling grip while capturing the image will lead to the image smudging. Such smudging may be resolvable algorithmically. Or if algorithmic resolution is not possible, the user may be prompted via the smart device to retake the image.

The smart device model is queryable using most modern smart device APIs. Algorithms can be tuned for the cameras on specific smart devices. The most important aspects that will change with smart device will be camera projection parameters, color filter spectra, flash spectrum and image processing algorithms. Data may be gathered regarding mobile-phone-model-specific parameters can be tweaked and pushed to all similar smart devices.

The smart device 140 may record data, such as the time, date, and geolocation of the test. All of this data is stored in a unique test results file referenced by the unique test number that is associated with that device’s EEPROM ID. User 105 can then send this test file via Wi-Fi or cellular network to any database or individual (for example, his/her doctor, the public health authority, etc.). User 105 may also download the result as a PDF file for future reference. User 105 can simply check off the anonymous box if they want to share the results but not have it linked to his/her identity. This is all achieved via a few simple clicks of mobile app 142 on smart device 140. Further, a centralized database of test results may be maintained on networked computer 160 to track epidemiologically relevant information, such as distribution of individuals with positive/negative test results.

### Additional Test Cartridge Details

FIG. 13 is another photo of the test cartridge 805 shown in FIG. 9A and FIG. 9B. Again, test cartridge 805 may be an example of the test cartridge 110 of system 100. In this photo, the three reagent blister pack wells 810 and the two-port well 813 are populated with blister packs 809. Further, a sample collection device 807 is coupled to the sample input port 812 supplying two-port well 813. Sample collection device 807 may be, for example, a syringe with or without a collection tube at its tip.

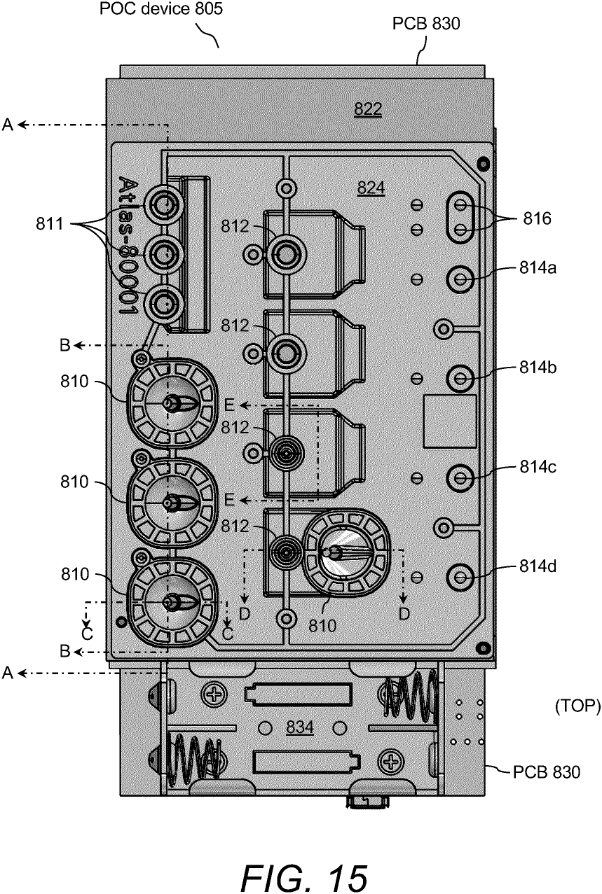

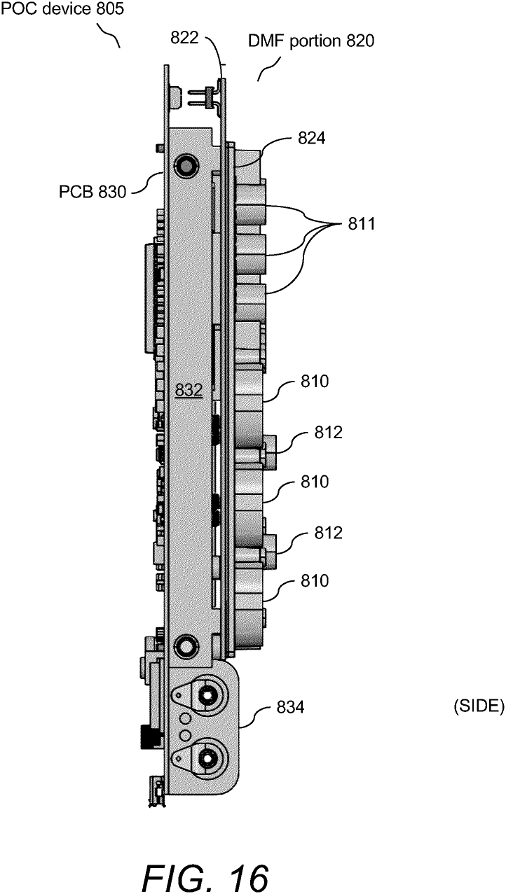

Test cartridge 110 (e.g., test cartridge 805) may include a DMF cartridge portion (e.g., PCB bottom substrate and plastic top substrate separated by a gap) and a control electronics PCB integrated together in one assembly. By way of example, FIG. 14 is a perspective view showing more details of test cartridge 805. Further, FIG. 15, FIG. 16, and FIG. 17 show a top view, a side view, and a bottom view, respectively, of test cartridge 805 shown in FIG. 14. Again, test cartridge 805 may include three reagent blister pack wells 810 for holding blister packs 809, three reagent input ports 811, four sample input ports 812, four sample detection spots 814 (e.g., 814a, 814b, 814c, 814d), and two controls detection spots 816 (e.g., one positive and one negative control). Sample input ports 812 may be, for example, luer port or simple well-ports. Again, one sample input port 812 may be one input of two-port well 813 and a blister pack 809 may be the second input of two-port well 813. FIG. 14 shows sample collection device 807 coupled to the sample input port 812 supplying two-port well 813. Further, each of the reagent blister pack wells 810 and two-port well 813 may include a burst point feature for bursting a blister pack 809 (see FIG. 23 and FIG. 24).

Further, test cartridge 805 may include a DMF portion 820 and a control electronics PCB 830 held in relation to one another via an assembly frame 832. Test cartridge 805 may also include a battery pack 834 for holding a pair of AA or AAA batteries. DMF portion 820 provides DMF capabilities for processing of biological materials. DMF portion 820 may be used, for example, to perform sample preparation. DMF capabilities of DMF portion 820 may generally include, but are not limited to, transporting, merging, mixing, splitting, dispensing, diluting, agitating, deforming (shaping), and other types of droplet operations. DMF portion 820 utilizes the principle of electrowetting to directly manipulate liquid droplets. DMF portion 820 may include, for example, a PCB bottom substrate 822 and a top substrate 824 separated by a droplet operations gap (not shown).

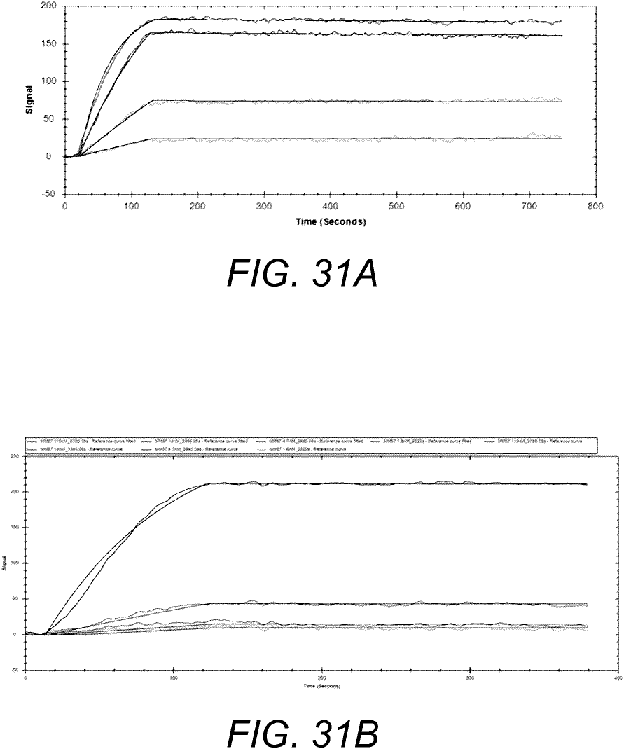

PCB bottom substrate 822 may be a standard PCB. Generally, PCB bottom substrate 822 may include, for example, droplet operations electrodes (e.g., electrowetting electrodes, see FIG. 31) and/or one or more dielectric layers to form a droplet operations surface. Top substrate 824 may be, for example, a glass or plastic substrate that may be substantially transparent to light.

While certain electrical components may exist on PCB bottom substrate 822, any other control electronics (see FIG. 17) needed in test cartridge 805 may be installed on control electronics PCB 830. Accordingly, various electrical connections may exist between control electronics PCB 830 and PCB bottom substrate 822 of test cartridge 805.

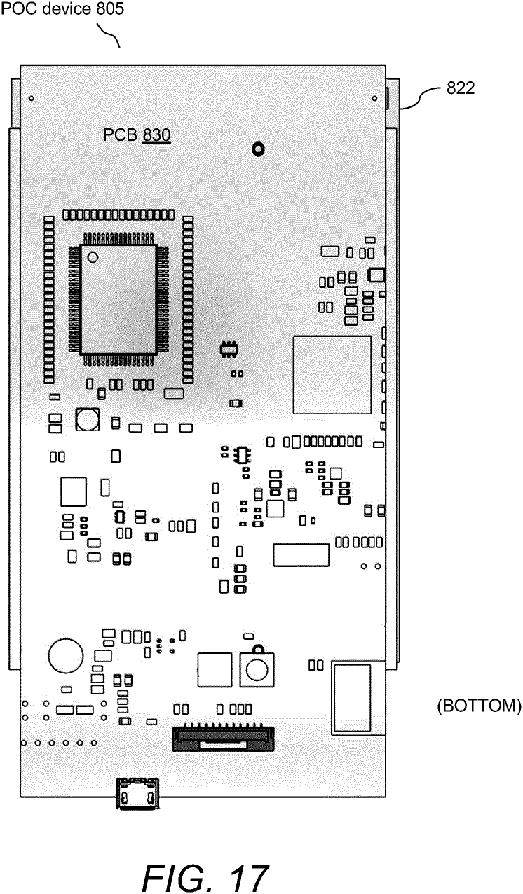

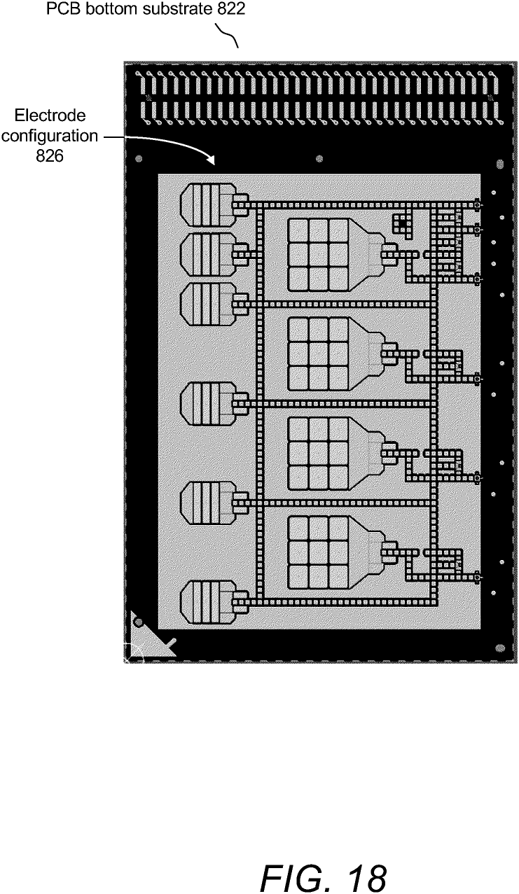

FIG. 18 is a plan view of an example of PCB bottom substrate 822 of test cartridge 805 shown in FIG. 14. PCB bottom substrate 822 may include, for example, an electrode configuration 826. In this example, electrode configuration 826 may include lines, paths, and/or arrays of droplet operations electrodes and reservoir electrodes that correspond to the arrangement of reagent blister pack wells 810, reagent input ports 811, sample input ports 812, two-port well 813, sample detection spots 814 (e.g., 814a, 814b, 814c, 814d), and controls detection spots 816 shown in FIG. 13 through FIG. 17.

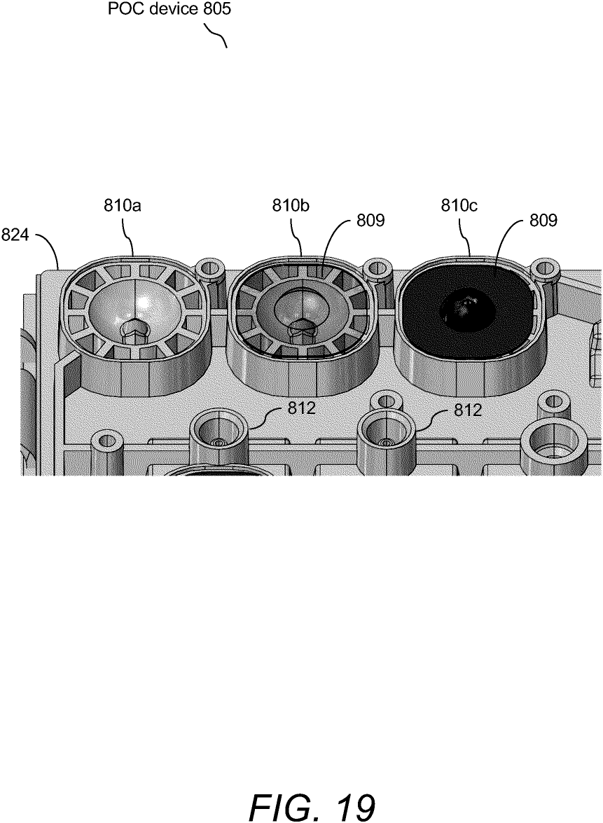

FIG. 19 is a perspective view of the portion of test cartridge 805 shown in FIG. 14 that includes the three reagent blister pack wells 810. In this example, reagent blister pack well 810a is not populated with a blister pack 809, reagent blister pack well 810b is populated with a substantially transparent blister pack 809, and reagent blister pack well 810c is populated with a standard blister pack 809. Radial features of each reagent blister pack well 810 provide a surface for supporting the blister pack 809. The design of the reagent blister pack wells 810 is such that thick-wall defects may be avoided when formed by injection molding.

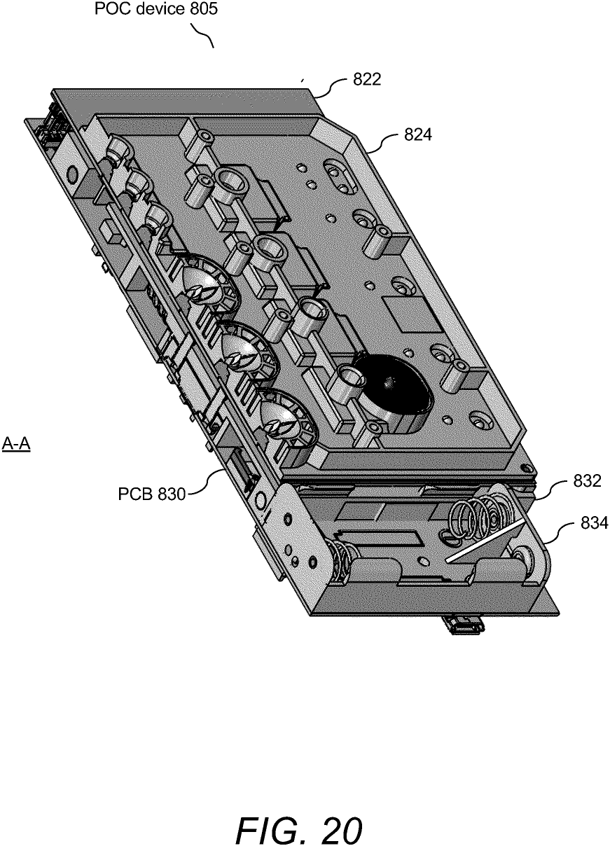

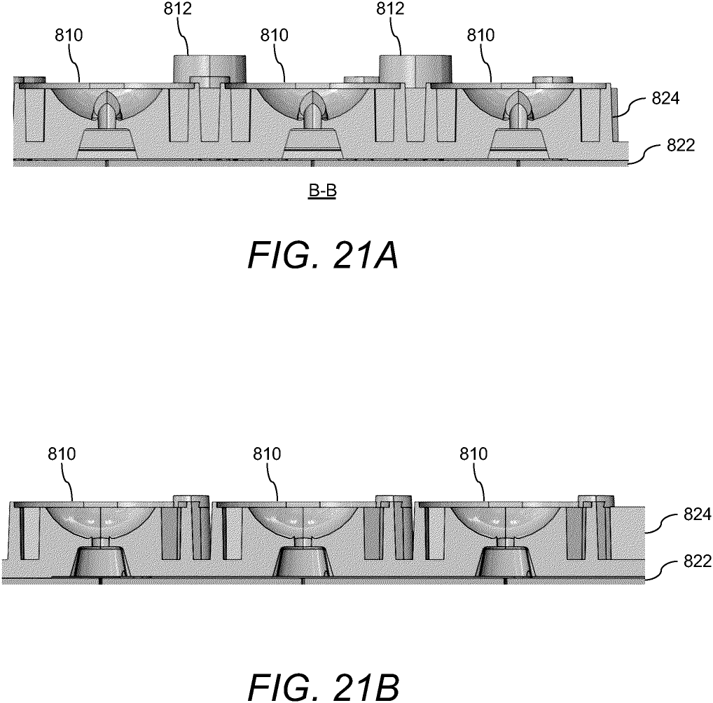

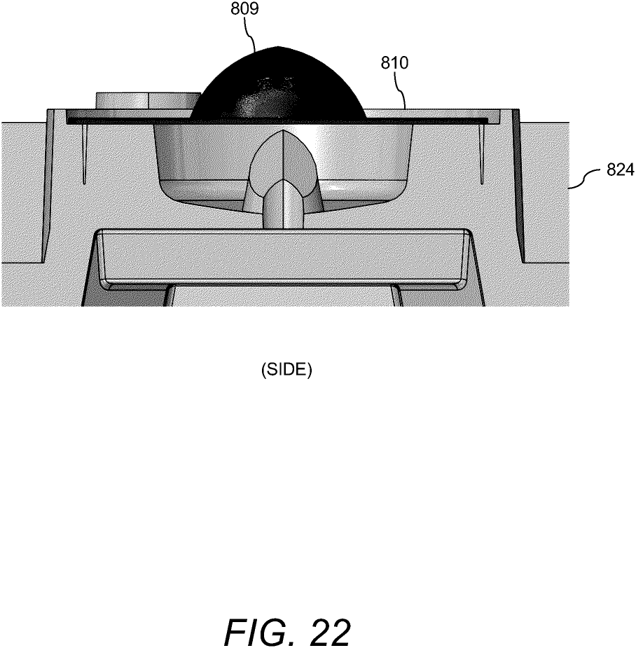

FIG. 20 is a cross-sectional view of test cartridge 805 taken along line A-A of FIG. 14. Further, FIG. 21A shows a cross-sectional view of test cartridge 805 taken along line B-B of FIG. 14, which is the arrangement of the three reagent blister pack wells 810. FIG. 21B is the reverse view of FIG. 21A. Further, FIG. 22 shows a side view of one reagent blister pack well 810, while FIG. 23 shows a cross-sectional view of test cartridge 805 taken along line C-C of FIG. 14, which is one reagent blister pack well 810. FIG. 23 show that each reagent blister pack well 810 may include a reagent port 840 leading from the blister pack 809 to the well below. Additionally, each reagent blister pack well 810 may include a burst point feature 842 for bursting the blister pack 809. Burst point feature 842 may be a pointed or sharp-edged and sloped feature. For example, when pressure is applied to the top of the blister pack 809, the foil seal on the underside (not shown) pushes down and is pierced or punctured by burst point feature 842. A hydrophobic coating on burst point feature 842 and the slope assists to guide the fluid to drop into the dispenser portion (e.g., a DMF dispense region 844) of reagent blister pack well 810.

FIG. 24 is a cross-sectional view of test cartridge 805 taken along line D-D of FIG. 14, which is two-port well 813. Again, a sample input port 812 may be one input of two-port well 813 and a blister pack 809 may be the second input of two-port well 813. Sample input port 812 may be, for example, a luer port or simple well-port. Both sample input port 812 and blister pack 809 supply a reservoir 846. Reagent port 840 leads from the blister pack 809 to reservoir 846. Generally, two-port well 813 provides a blister pack and sample combination by integrating both sample input and a microfluidic blister into the same feature.

A main benefit of two-port well 813 is that it provides a way to mix two large volumes in a DMF environment. Following mixing, the analyte may be concentrated into a small volume that may be eluted for downstream analysis. In addition to providing an elegant method of bridging the volume divide between the benchtop (10-100 µL) to DMF (<1 µL), in two-port well 813 the two features complement each other by being an air/oil pressure release point to prevent bubbles being introduced into the device. For example, sample input port 812 (e.g., luer port) may be the air vent for the blister bursting, once blister pack 809 is burst.

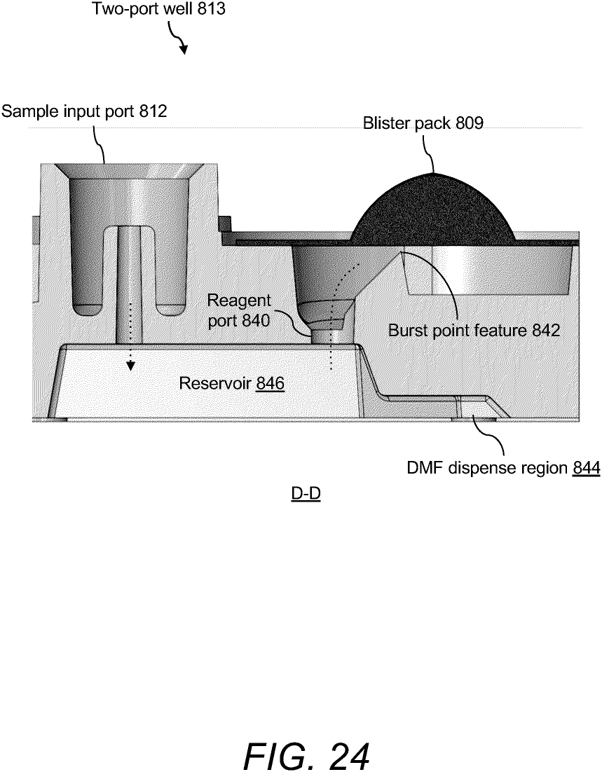

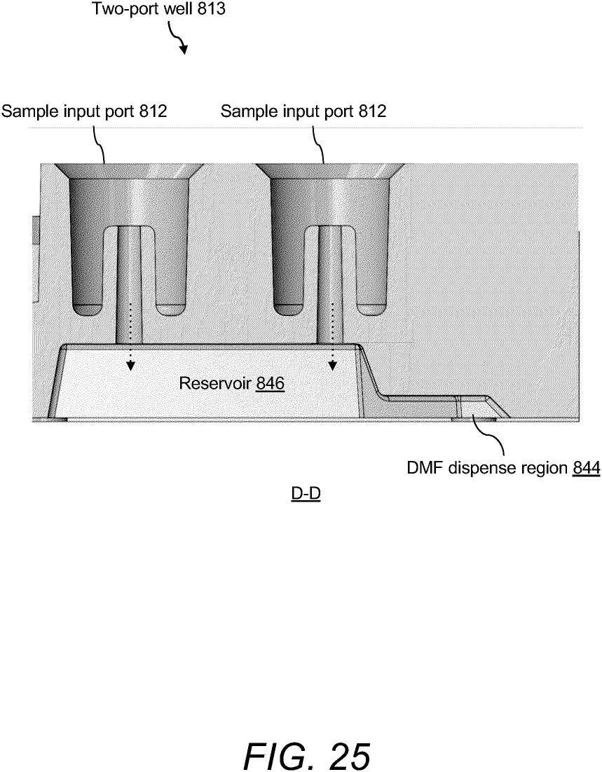

FIG. 25 is a cross-sectional view showing another example of two-port well 813. The two-port well 813 shown in FIG. 25 is substantially the same as the two-port well 813 shown in FIG. 24 except that the blister pack 809 is replaced by a second sample input port 812 (e.g., luer port). That is, the two ports of two-port well 813 is a pair of sample input ports 812 (e.g., luer ports).

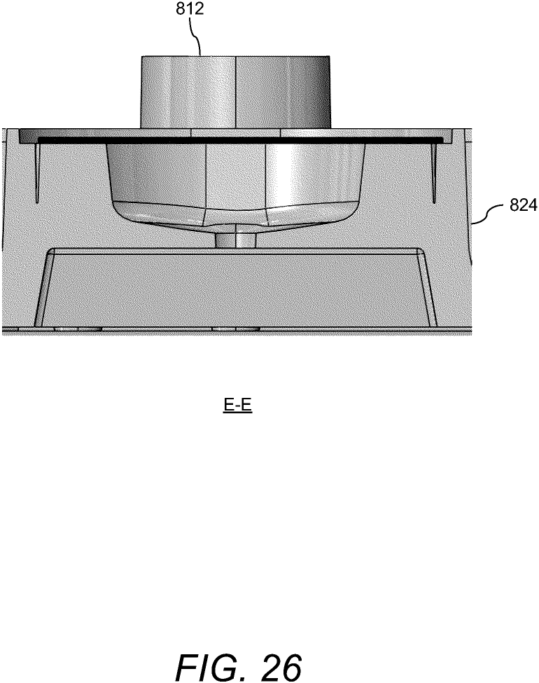

FIG. 26 is a cross-sectional view of test cartridge 805 taken along line E-E of FIG. 14, which is the well adjacent to two-port well 813 that is supplied by a different sample input port 812.

Color filters can be used to increase the accuracy. Consider, for example, two color filters that produce the same outcome for a negative presence but different outcomes for a positive presence. The color contrast between these two-color filters will then be indicative of the presence α. Color filters can help detection less dependent on the color filters in the camera by providing saturated color filters. The color filters may also be created to be narrow band enough, so as to produce a very low-resolution spectroscope.

The above technique can be applied even if a more complex optical model (one that accounts for multi-path light conduction and other optical scattering peculiarities) is used. The assay optical model can be generated by careful spectral data collection of actual assays rather than any physics-based approximation. To gain further accuracy, such an optical model need not be linearized. It could be used in fully nonlinear mode. Alternatively, instead of linearizing the model, a higher-order Taylor series approximation may be used.

A smart device flash can be used to overcome bad color rendering qualities (CRI) of natural illumination. Though flashes can have different illumination spectra, most modern camera designers ensure that the flash spectrum is broad, and the CRI is decent. Multiple pictures may also be used to improve rendering.

More than one reference patch of the same reflection spectrum may be placed on the device at various locations. Apart from making the pattern easier to recognize in the image, this design will have the advantage of testing the variation in ambient, flash and stray light in various locations of the image. If the variation is high and cannot be modeled as a gradual change from one part of the image to another, the captured images may be deemed to be too ill-conditioned for a confident measurement of the assay, and the system may be programmed to produce a user prompt guiding a user to capture a better image. This prompt may include advice such as “use sunlight, avoid fluorescent light, avoid shadows, avoid glares”, etc.

An anti-reflective coating may be used to reduce Fresnel reflections and thus the stray light.

### Other Improvements

The photograph may be captured using a smart device, such as a handheld smart device, such as a smartphone. This will cause a variation in where the assay and reference patch elements occur in the captured image. The present disclosure may make use of image registration techniques (perspective projection registration techniques with unknown projection and camera parameters are well known corrections for common lens distortions have also been investigated in the literature) to identify elements of the image. If the required elements cannot be found or are too distorted, user prompts can be used to help the user acquire a better image. Alternatively, a video capture mode may be used for recording the image, and the best images from a sequence of captured images may be automatically chosen.

Apart from the assay and a pattern of reference patches, the device may also include a graphical code (such as a QR code) identifying itself. This can be used to identify the device generation and optimize the algorithm accordingly.

### Target Analytes

Any analyte detectable by immunoassay may be the subject of a test of the present disclosure. Examples include viruses and bacteria. Examples of viruses include arboviruses, flaviviruses, alphaviruses, herpesviruses, papillomaviruses, picornaviruses, polyomaviruses, retroviruses, respiratory viruses such as influenza virus, respiratory syncytial virus, parainfluenza viruses, metapneumovirus, rhinovirus, coronaviruses, adenoviruses, and bocaviruses; rhabdoviruses, and rotaviruses.

### EXAMPLES

### Kinetics of SARS-CoV-2 Antibody & Spike Protein RBD

To demonstrate binding kinetics and detection of SARS-CoV-2 antibody to its cognate viral target, SARS-CoV-2 spike protein receptor binding domain (RBD), a direct binding assay was performed using the OpenSPR-XT™ instrument and OpenSPR Carboxyl sensor (available from Nicoya, Kitchener, ON, Canada). The binding assay used the SARS-CoV-2 RBD protein as ligand (available from Sino Biological, CAT#: 40150-V08B2) and a Rabbit Anti-Spike Protein Monoclonal Antibody (mAB) (available from Sigma, CAT#: SAB3700861-2MG) as the primary antibody. The ligand was immobilized on the carboxyl sensor surface using EDC/NHS coupling chemistry. The assay was performed in a normal buffer background (i.e., an analysis running buffer), as well as by spiking the antibody into a 50% serum solution. For the normal buffer background analysis, a solution of the primary antibody (anti-spike mAB) was prepared in an analysis running buffer (PBS-T + 1% BSA) at a concentration of 150 nM, and further diluted in 3-fold serial dilutions. A second solution of the primary antibody was prepared in a solution of diluted rabbit serum (available from Jackson Immunoresearch CAT#: 011-000-10; diluted 2-fold in analysis running buffer) at a concentration of 150 nM, which was further diluted in the analysis running buffer in 3-fold serial dilutions. Further, to optimize the limit of detection (LOD) for the primary antibody, a secondary antibody amplification process was used.

The assay procedure was as follows:

Following the start-up procedure in the software, the OpenSPR-XT™ instrument was set up, using phosphate buffer saline, 0.1% Tween (PBS-T) as the initial running buffer.

Sensor surface was prepared following the wizard steps in the OpenSPR™ software.

The ligand was immobilized on the EDC/NHS activated surface at a concentration of 10 µg/ml and a flow rate of 20 µL/min on channel 2 only (designated as the response channel).

Bovine serum albumin (BSA) was immobilized on both channels as a blocker at a flow rate of 20 µL/min.

Remaining COOH groups were blocked with the OpenSPR™ blocking solution.

The instrument was primed in PBS-T + 1% BSA as the analysis running buffer.

The purified primary antibody was prepared in the analysis running buffer at a concentration of 150 nM, and further diluted in 3-fold serial dilutions.

The rabbit serum was diluted 2-fold in the analysis running buffer and used to prepare a 150 nM sample of the primary antibody, which was further diluted in analysis running buffer in 3-fold serial dilutions.

The secondary antibody samples were prepared in the analysis running buffer at a concentration of 150 nM and further diluted in 3-fold serial dilutions.

The primary antibody was injected over the ligand at 50 µL/min (2-min association, 5-minute dissociation) in order of increasing concentration.

The secondary antibody was injected over the primary antibody at 50 µL/min (2 min association, 5 min dissociation) at a constant concentration of 150 nM.

The ligand was regenerated with an injection of pH 1.5 glycine-HCl at 150 µL/min before each subsequent primary antibody injection.

Steps 10-12 were repeated for the primary antibody in diluted serum samples.

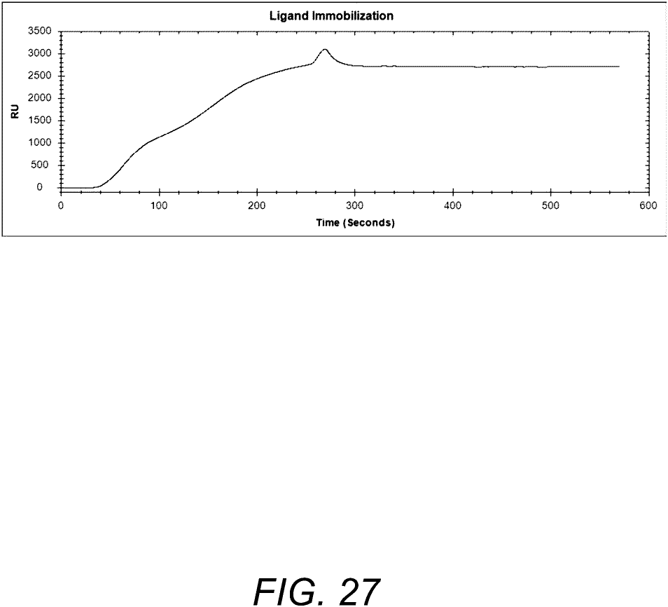

FIG. 27 is a plot showing the immobilization of the SARS-CoV-2 spike protein RBD on an OpenSPR-XT carboxyl sensor. The data show approximately 2500 RU of immobilization for the SARS-CoV-2 spike protein RBD.

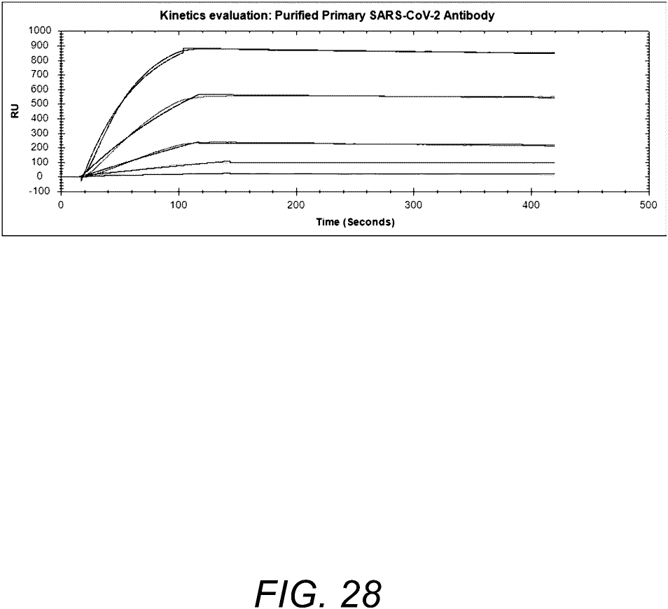

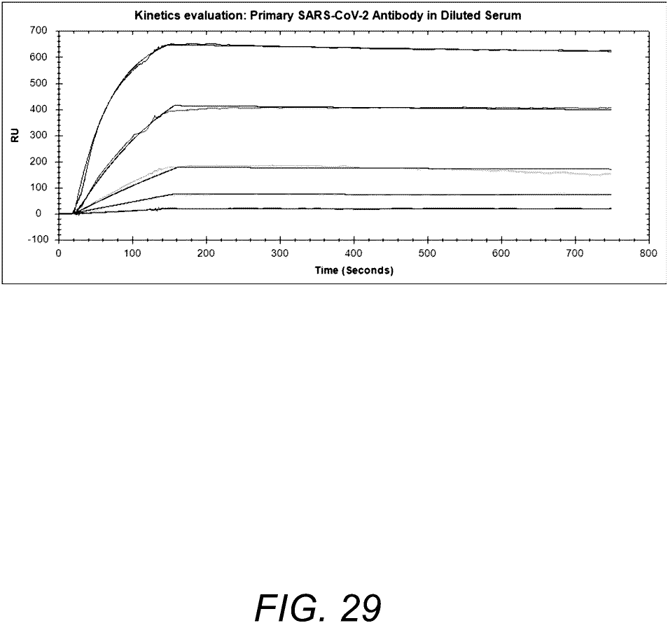

FIG. 28 is a plot showing the binding and kinetic fits of the SARS-CoV-2 primary antibody to the immobilized receptor domain (i.e., spike protein RBD). The data was fit to a one-to-one binding model using the TraceDrawer™ Kinetic Analysis Software (available from Ridgeview Instruments, Uppsala, Sweden). FIG. 29 shows the binding of the primary antibody in analysis running buffer to the immobilized ligand at concentrations of 150 nM, 50 nM, 16.7 nM, 5.56 nM and 1.85 nM. The solid black line represents the one-to-one kinetic model fits.

FIG. 29 is a plot showing the binding and kinetic fits of the rabbit serum diluted SARS-CoV-2 primary antibody to the immobilized receptor domain (i.e., spike protein RBD). The data was fit to a one-to-one binding model using the TraceDrawer™ Kinetic Analysis Software. FIG. 29 shows the binding of the primary antibody in 50% serum to the immobilized ligand at concentrations of 150 nM, 50 nM, 16.7 nM, 5.56 nM and 1.85 nM. The solid black line represents the one-to-one kinetic model fits.

The calculated kinetic constants for the binding experiments described with reference to FIG. 28 and FIG. 29, are shown in Table 1. FIG. 28, FIG. 29, and Table 1, the data show that similar values were obtained for the antibody samples diluted in serum, and the antibody samples prepared in the analysis running buffer. The data also demonstrates OpenSPR-XT’s ability to detect binding and measure kinetics for antibodies in serological (serum) samples.

TABLE 1

Kinetic values measured using OpenSPR™ with the TraceDrawer™ analysis software

| | | |
|---|---|---|
| Parameter | SARS-CoV-2 mAb in buffer | SARS-CoV-2 mAb in Serum |
| ka [1/M*s] | 1.40e5 (±2.27e0) | 1.30e5 (±3.94e0) |
| kd [1/s] | 1.42e-4 (±6.00e-6) | 6.75e-5 (±2.00e-5) |
| KD [M] | 1.01e-9 (±4.29e-11) | 5.18e-10 (±1.53e-10) |

For applications in diagnostics, the ability to measure antibody concentrations similar to those found in a serological sample is important. Secondary antibody amplification was therefore used to demonstrate a method to further improve the LOD for the primary antibody. In this technique, multiple secondary antibodies bind to the Fc region of a single primary antibody. This increased avidity leads to a secondary antibody binding signal that is larger than that of the primary antibody, and thereby provides an improved LOD of the primary antibody.

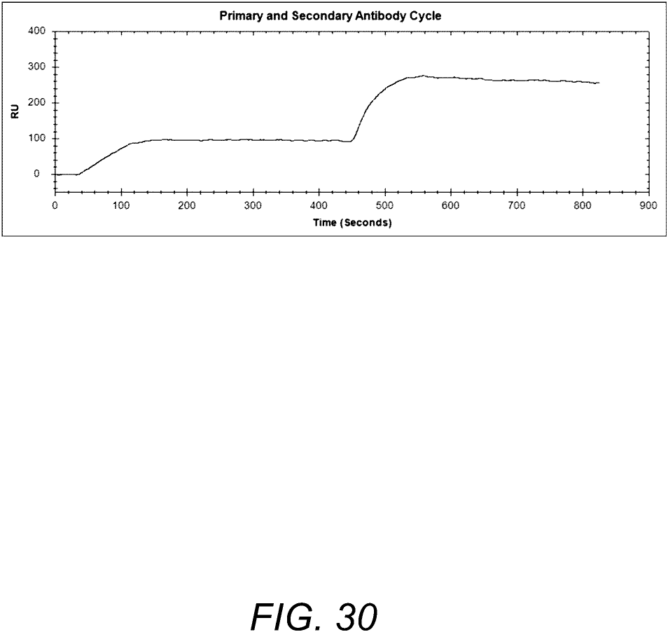

FIG. 30 is a plot showing a primary antibody plus secondary antibody amplification cycle. In this example, the primary antibody concentration was 5.56 nM. The secondary antibody response was plotted against the primary antibody concentration for each curve and fitted using a logarithmic model. The data show that secondary antibody binding to the primary antibody yields a signal that is about twice as high as that of the primary antibody alone. This fit determined that by using secondary antibody amplification, concentrations of the primary antibody could be detected into the picomolar range, yielding a tenfold improvement in LOD for the primary antibody compared to the direct assay.

Referring now to the plots shown in FIG. 27 through FIG. 30, the data demonstrate that the OpenSPR-XT™ technology can be used to measure binding kinetics of SARS-CoV-2 mAb to its cognate viral target in serological samples, with similar results to those obtained using an antibody sample prepared in an analysis running buffer. The data also show that the presence of the SARS-CoV-2 mAB can be directly detected in low nanomolar concentrations, and that this LOD can be improved tenfold, down to picomolar concentrations, via secondary antibody amplification.

### ELISA Assay

To achieve parity with molecular based assays (e.g., RT-PCR), clinical research indicates a limit of detection between 3,000 to 30,000 viral copies per milliliter to detect SARS-CoV-2 within the first 72 hours of symptom onset. This would translate to approximately 1.0 - 10 fM of viral spike protein equivalent, which represents one potential viral target for assay development.

In one example, detection of a viral antigen may be performed using a sandwich ELISA technique, wherein a target analyte antigen (e.g., the SARS-CoV-2 spike protein RBD) is captured on a reaction surface using a capture antibody and then detected using a detection antibody and a reporter system.

To evaluate suitable antibody combinations for detecting SARS-CoV-2 using a sandwich ELISA approach, antibodies were first screened for affinity and kinetics to the SARS-CoV2 spike protein using the OpenSPR-XT™ instrument and carboxyl sensor. Tests to determine affinity were conducted on four antibodies obtained from Sino Biological: MM57, MM42, R001, and D001 (HRP-labeled). Briefly, SARS-CoV-2 spike protein was immobilized onto a carboxyl sensor using EDC/NHS coupling chemistry. Antibodies were injected over the immobilized SARS-CoV-2 spike protein at different concentrations.

FIG. 31A is a plot 1700 showing an example of an SPR assay result for the R001 antibody. Table 2 includes a summary of the affinity results. Antibody concentrations were yellow line = 1.2 nM, green line = 3.4 nM, blue line = 11 nM, and red line = 33 nM. The data show that all four antibodies tested provide a high affinity for antigen binding on the nM or pM level.

TABLE 2

SPR results for 4 different SARS-CoV-2 antibodies binding to the spike protein

| | | | |
|---|---|---|---|
| | kon (1/M*s) | koff (1/s) | KD |
| MM42 | 8.92e4 | 5.93e-4 | 6.65 nM |
| MM57 | 1.07e5 | 5.69e-5 | 534 pM |
| R001 | 1.26e6 | 7.74e-5 | 60.9pM |
| D001 | 1.76e5 | 1.70e-7 | 1.05pM |

The antibody pairs were also tested for epitope overlap using SPR analysis. FIG. 31B is a plot showing the epitope overlap for the MM57 and MM42 antibodies. In this example, MM42 saturated the capture sites on the sensor. MM57 was then injected onto the sensor and binding was observed, indicating that the antibodies bind to different epitopes.

The epitope mapping results are summarized in Table 3. These results were used to inform which antibodies could be used as pairs in a sandwich ELISA protocol.

TABLE 3

Epitope mapping of SARS-CoV-2 antibodies binding to the spike protein

| | | | | |
|---|---|---|---|---|
| | MM57 | MM42 | R001 | D001 |
| MM57 | n/a | non-overlapping | potential overlap | not tested for overlap |
| MM42 | non-overlapping | n/a | potential overlap | not tested for overlap |
| R001 | non-overlapping | not tested for overlap | n/a | non-overlapping |
| D001 | not tested for overlap | not tested for overlap | non-overlapping | n/a |

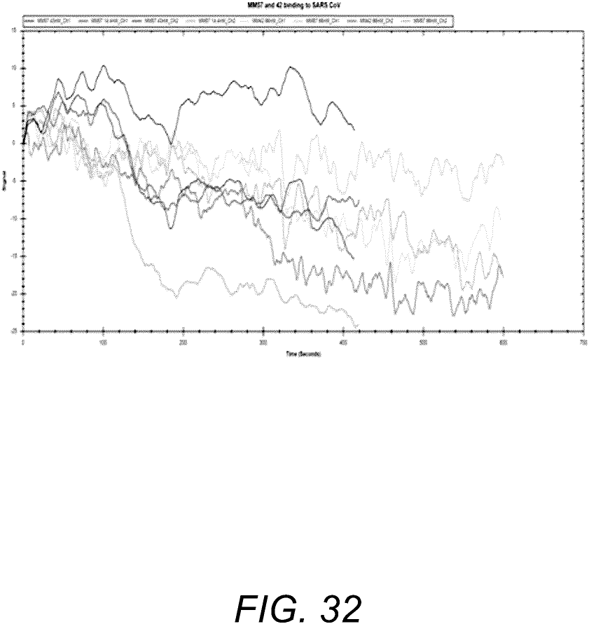

SPR tests were also performed to confirm antibody specificity by testing the antibodies against SARS-CoV-1 and MERS spike proteins. FIG. 32 is a plot showing a representative example of a cross reactivity study using MM57 and MM42 antibodies against SARS-CoV-1 spike protein using SPR. The data show that no response was observed indicating that no cross reactivity was detected. The cross-reactivity results for MM57, MM42, and R001 are summarized in Table 4. Note that D001 was not tested in the cross-reactivity study as it is not intended to be used as a specific capture antibody.

TABLE 4

Cross-reactivity of antibodies to SARS-CoV-1, MERS, and Sars-Cov-2

| | | | |
|---|---|---|---|
| | MM57 | MM42 | R001 |
| SARS-CoV-1 | no specificity (or reactivity) | no specificity (or reactivity) | no specificity (or reactivity) |
| MERS | no specificity (or reactivity) | no specificity (or reactivity) | no specificity (or reactivity) |
| SARS-CoV-2 | specificity | specificity | specificity |

Based on the above experimental observations, MM57 and R001 were selected as the best performing antibodies for further assay development.

A custom biotinylation process using a long PEG tether was developed in house in order to biotinylate the antibodies (MM57 and R001) for use on streptavidin coated plates and beads. The affinity and kinetics of the biotinylated antibodies were measured as described above with reference to plot of FIG. 31A and Table 2 to ensure that the biotinylation process had no impact on the function of the antibodies. The results for the biotinylated antibodies MM57 and R001 are shown in Table 5. The results show that both antibodies performed similarly to pre-biotinylation.

TABLE 5

SPR results for MM57- and R001-biotinylated antibodies binding to the spike protein

| | | | |
|---|---|---|---|
| | kon (1/M*s) | koff (1/s) | KD |
| MM57-biotinylated | 1.09e5 | 9.91e-5 | 905 pM |
| R001-biotinylated | 6.40e5 | 3.30e-5 | 51.6 pM |

Biotinylation was also confirmed by using SPR. Briefly, antibodies were captured on the sensor surface using immobilized spike protein. Streptavidin was then introduced to the sensor, wherein a positive signal indicated a successful biotinylation (data not shown).

To further develop assay conditions, well-plates coated in streptavidin and maleic anhydride were used to test various conditions and perform control tests. A variety of conditions with both antibodies (MM57 and R001) in various capture and reporter configurations were tested and substantial controls were performed to develop a proof-of-concept plate-based ELISA to guide the rest of the assay development. The finalized assay used biotin-R001 immobilized on a streptavidin plate. Various concentrations of spike protein were plated in separate wells (in PBS-T+1% BSA buffer). After washing, the D001 antibody labeled with HRP at a 10,000X dilution factor was added as the reporter antibody. Following washing, the HRP substrate TMB was added for a 20-minute incubation period followed by the addition of 1 M HCl as a stop solution.

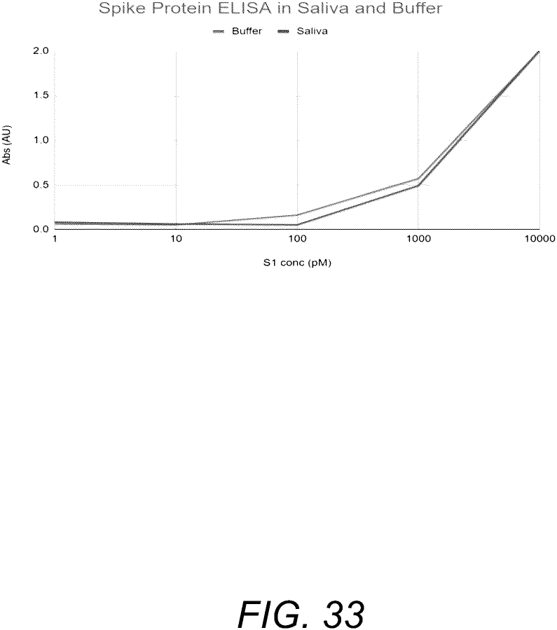

FIG. 33 is a plot showing the absorbance readout at 450 nm for the ELISA plate assay using R001 to capture spike protein diluted into either buffer or saliva. In this example, D001 labeled with HRP was used as the secondary antibody for readout with TMB. The data demonstrate detection of less than 100 pM of spike protein. LODs as low as 0.1 pM of spike protein have been detected with this assay format. The data also show that the assay performed similarly in both saliva and buffer.

The ELISA results demonstrate the selected antibodies and assay conditions developed provide a framework for COVID-19 antigen testing. To detect SARS-CoV-2 with similar accuracy to molecular based assays, the LOD was further improved with bead-based assays.

### Magnetically Responsive Bead Assays

In order to improve the assay LOD and integrate the assay into a DMF environment, bead-based assays were also developed. The bead-based assays include both magnetically responsive beads for capture and concentration of the virus, and latex beads for enhancing the LOD.

Magnetically responsive beads that are coated with the primary antibody enable amplification of the virus concentration up to 1,500X from a saliva sample, for example, by concentrating the virus from a 500 µL sample to a 330 nL sample. For example, the magnetically responsive beads can be dispersed into the saliva sample and then pelleted (e.g., using a magnet) and resuspended into a single droplet unit (DU) of 330 nL. Magnetically responsive beads also enable efficient on-cartridge washing, significantly improving the quality and performance of the assay. Four different streptavidin coated magnetically responsive beads from 4 different suppliers were tested to determine relative loading densities using biotin-HRP. The results are shown in Table 6. The data show that the ClickChem magnetically responsive beads had the highest HRP loading relative to the other magnetically responsive beads tested.

TABLE 6

Biotin-HRP loading levels on 4 different streptavidin coated magnetically responsive beads measured using TMB readout

| Bead Source | HRP Loading (nmol/mg) |
|---|---|
| Ray Biotech | 0.00827 |
| Eurofins Abraxis | 0.01485 |
| Trilink Vector Labs | 0.10397 |
| ClickChem | 0.12010 |

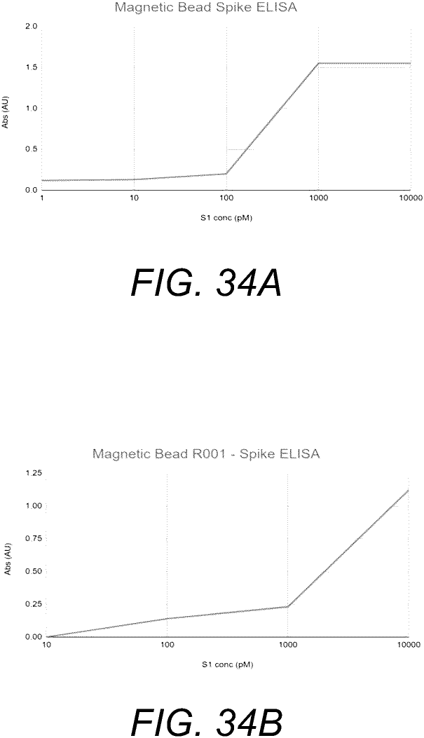

For initial assay development and verification, spike protein was biotinylated and captured using the streptavidin coated beads. FIG. 34A is a plot showing sub 100 pM detection of biotinylated spike protein captured with streptavidin coated magnetically responsive beads. D001 (HRP labeled) was used as the secondary reporter antibody and results read out using TMB. The show that the assay was successful in detecting spike protein at concentrations below 100 pM.

The plate-based ELISA using R001 capture antibody (described with reference to FIG. 34) was then transferred to streptavidin coated magnetically responsive beads. Biotinylated R001 antibody was immobilized on the streptavidin coated magnetically responsive beads and incubated with various concentrations of spike protein. D001 (HRP labeled) was used as the secondary antibody for readout with TMB. FIG. 34B is a plot showing sub 100 pM detection of spike protein using R001 coated magnetically responsive beads to capture spike protein. Note that there was likely bead loss due to washing, so the LOD is anticipated to be well below this with improved washing and handling protocols. Additionally, pre-concentration was not done with the magnetically responsive beads, so using the DMF platform we expect this LOD can be improved by up to 1,500X putting the estimated LOD into the femtomolar range. From these observations, it was concluded that the magnetic bead assay was successful at detecting spike protein at a relevant concentration(s) for a COVID-19 assay.

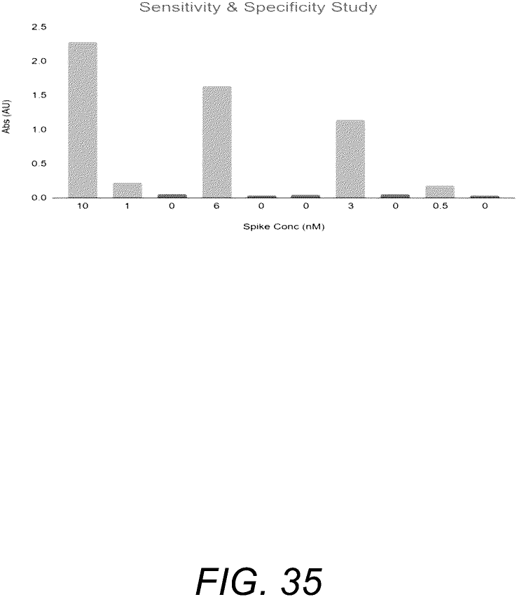

To test the sensitivity and specificity of the magnetic bead ELISA assay, 10 samples were prepared (5 negative and 5 positive) with spike protein in saliva. FIG. 35 is a plot showing the correct identification of all saliva samples as either positive or negative for spike protein. The spike concentrations (nM) for each saliva sample is shown on the x-axis. All positive samples have signals that were at least 3X above the average of the 5 negative (0 nM spike protein) samples. The ELISA assay correctly identified all 10 samples, indicating 100% sensitivity and 100% specificity in this limited study.

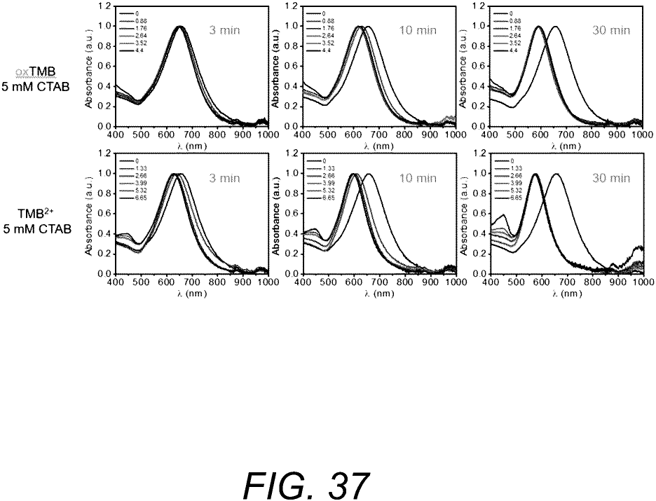

Once the magnetic bead ELISA was validated, the assay was repeated using inactive SARS-CoV-2 (sequence: hCoV-19/Canada/ON-VIDO-01/2020). FIG. 37A is a plot 2200 showing the results from the magnetic bead ELISA on inactive SARS-CoV-2 virus. Three concentrations were tested: 1X, 10X and 100X dilutions (stock concentration of 1.2*10^6 pfu/ml). The data show that the inactive virus was successfully captured and detected using the R001 functionalized magnetically responsive beads with D001 (HRP labeled) as the reporter antibody.

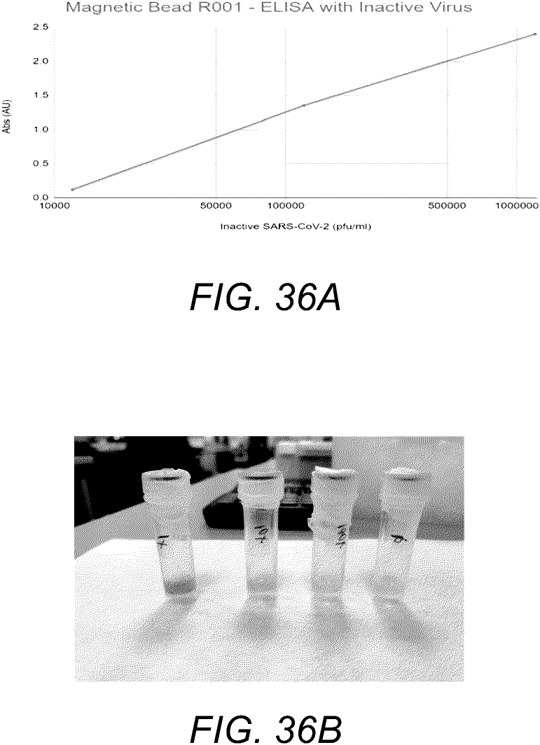

FIG. 36B is a photo of the ELISA results indicated in the of FIG. 37A and showing that all three concentrations of virus could be visually detected relative to the control sample. The tubes are shown from highest to lowest viral concentration from left to right, i.e., 1X, 10, and 100X dilutions from a stock of 1.2e6 pfu/mL.

Referring now to plot 2200 of FIG. 37A and photo 2205 of FIG. 37B, the data show that the magnetic bead assay can be used to detect SARS-CoV-2 at concentrations typical of the LOD for molecular based assays. To further improve the LOD, a dual-bead assay can be used to improve the LOD by up to 5 orders of magnitude (not shown).

### Plasmonic ELISA

We have demonstrated that our assay can reach the required LOD when using an absorbance spectrophotometer for the final read out. To circumvent the need for using an absorbance spectrophotometer for the final assay readout, a visual readout approach was developed using a plasmonic nanostructure that changes color due to etching caused by an oxidized TMB substrate that is produced during the enzymatic reaction with HRP. After extensive optimization, including nanoparticle type, size, shape, concentration, reaction time, TMB type, surfactants, buffer, and pH, we successfully developed a colorimetric readout that can be read visually or through the use of our custom developed smart device, such as a smartphone, camera application. The etching results of 4 different nanostructures are summarized in Table 7. Nanostars and nanorods did not show significant etching. Nanospheres and nanourchins did show etching in both TMB+ and TMB2+.

TABLE 7

Etching results of 4 different nanostructures in TMB+ and TMB2+ at 3, 10, and 30 minutes of incubation

| | | | | | | |
|---|---|---|---|---|---|---|
| | TMB+ | | | TMB2+ | | |
| Time (min) | 3 | 10 | 30 | 3 | 10 | 30 |
| Nanourchins | etching of positive samples only | etching of positive samples only | etching of negative control observed | etching of positive samples only | etching of positive samples only | etching of negative control observed |
| Nanostars | no etching | no etching | no etching | no etching | no etching | no etching |
| Nanorods | no etching | no etching | no etching | no etching | no etching | no etching |
| Nanospheres | etching of positive samples only | etching of positive samples only | etching of positive samples only | etching of positive samples only | etching of positive samples only | etching of positive samples only |

FIG. 37 is a panel of plots 2300 showing UV-Vis spectra of nanourchins exposed to different concentrations of oxidized TMB+ or TMB2+ with 5 mM CTAB. Spectra were taken after 3, 10, and 30 minutes of incubation.

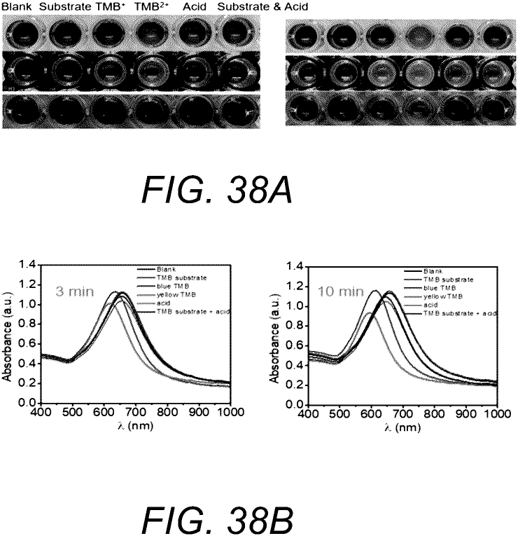

FIG. 38A is photographs and FIG. 38B shows UV-Vis spectral plots 2405 of gold nanourchins (AuNU) exposed to TMB and TMB2+ after 3 and 10 minutes of incubation. Control solutions (i.e., blank, TMB substrate only, acid only, and TMB substrate and acid) are also shown. The photographs were taken against white, black, and yellow backgrounds. Black and yellow are two background colors that may be used with a DMF device.

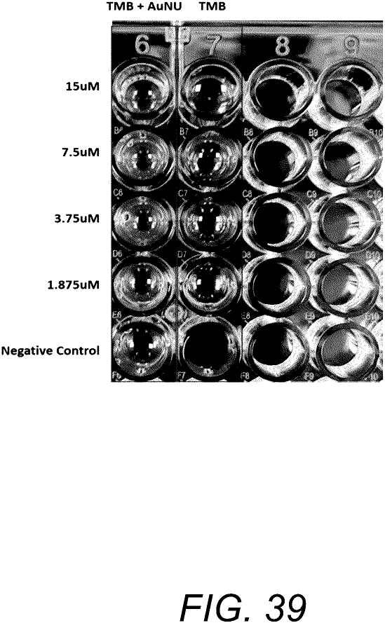

The optimal conditions were found with 90 nm gold nanourchins (AuNU) with an OD 3.5 and 15 mM CTAB. The plasmonic ELISA is substantially more sensitive for visual readout compared to TMB on its own. FIG. 39 is a photograph showing a comparison between the plasmonic ELISA and a conventional ELISA result over a range of oxidized TMB concentrations. Even at the highest concentration of 15 µM, the conventional ELISA result cannot be visually detected, whereas the plasmonic ELISA can be read out at the lowest concentration tested of 1.875 µM. With current conditions we have demonstrated visual readout down to 1 µM of oxidized TMB in <20 minutes of assay time using the DMF cartridge. This would translate to a visual LOD of ~5000 viral particles/mL.

Based on the observations described above, nanourchins (e.g., AuNU) were selected as an optimal candidate due to their enhanced visibility on the DMF cartridge.

Although the foregoing subject matter has been described in some detail by way of illustration and example for purposes of clarity of understanding, it will be understood by those skilled in the art that certain changes and modifications can be practiced within the scope of the appended claims.

What is claimed is:

1. A digital microfluidic (DMF) device for performing a self-test for a target analyte, the DMF device comprising:
a. a DMF cartridge comprising a bottom substrate and a top substrate separated by a droplet operations gap, wherein the bottom substrate comprises a plurality of droplet operations electrodes configured for performing droplet operations on a liquid droplet in the droplet operations gap;
b. one or more reaction chambers or reaction zones on the bottom substrate that are supplied by an arrangement of the droplet operations electrodes, wherein each reaction chamber or reaction zone comprises at least one detection spot and is configured for performing a plasmonic particle-assisted ELISA (pELISA) for detection and quantification of a target analyte in a sample droplet; and
c. a controller coupled to the electrodes and programmed to activate and deactivate the electrodes and thereby effect droplet operations for performing the self-test.

2. The DMF device of claim 0 wherein a and b are part of a cartridge and c is part of an instrument to which the cartridge is mounted.

3. The DMF device of claim 0 wherein the bottom substrate comprises a printed circuit board.

4. The DMF device of claim 0 wherein the bottom substrate further comprises one or more reservoir electrodes configured for supplying the one or more reaction chambers or reaction zones via the droplet operations electrodes.

5. The DMF device of claim 0 wherein the top substrate comprises a glass or plastic substrate that is substantially transparent to light.

6. The DMF device of claim 0 wherein the top substrate further comprises one or more input ports for receiving and supplying an input reagent or sample fluid, wherein the input ports are arranged in relation to the one or more reservoir electrodes on the bottom substrate.

7. The DMF device of claim 0 wherein the top substrate further comprises one or more reagent wells for receiving a reagent blister pack, wherein the reagent wells are arranged in relation to the one or more reagent reservoir electrodes on the bottom substrate.

8. The DMF device of claim 0 wherein the one or more reagent wells further comprise a reagent port arranged to permit flow of reagent fluids from a reagent blister pack into the well.

9. The DMF device of claim 0 wherein at least one reagent well comprises a two-port well comprising:
i. a first input port comprising a luer port or simple port well for receiving and inputting a sample fluid; and
ii. a second input port comprising a reagent blister pack well for receiving and inputting a reagent fluid.

10. The DMF device of claim 0 wherein the second input port further comprises a blister pack burst mechanism attached to the top plate in proximity to the input second port for bursting a reagent blister pack and releasing the reagent fluids.

11. The DMF device of claim 0 wherein the blister pack burst mechanism comprises a pointed or sharp-edged feature.

12. The DMF device of claim 0 wherein the two-port well comprises two sample input ports for receiving and inputting a sample fluid.

13. The DMF device of claim 0 wherein the top substrate further comprises one or more detection spots arranged in relation to the one or more reaction chambers and/or reaction zones on the bottom substrate for positioning a droplet for detection.

14. The DMF device of claim 0 wherein the controller comprises a microcontroller and/or a microprocessor.

15. The DMF device of claim 0 further comprising one or more thermal control mechanisms situated in sufficient proximity of the droplet operations gap to permit thermal control in the droplet operations gap for controlling the processing temperature in the DMF device.

16. The DMF device of claim 0 further comprising one or more magnets situated in sufficient proximity to the droplet operations gap to permit magnetic manipulation of magnetically responsive beads and/or particles in a droplet in the droplet operations gap.

17. The DMF device of claim 0 further comprising a power source electrically coupled to the plurality of droplet operations electrodes in the droplet operations gap for supplying power for performing droplet operations on a liquid droplet in the droplet operations gap.

18. The DMF device of claim 0 wherein the power source comprises a rechargeable or non-rechargeable battery.

19. The DMF device of claim 0 wherein the power source comprises a wired communications link.

20. The DMF device of claim 0 wherein the wired communications link comprises a USB charging cable of a smart device.

21. The DMF device of claim 0 further comprising a communications interface for connecting to the controller and exchanging test information from the at least one detection spot with a remote computer processing unit (CPU).

22. The DMF device of claim 0 wherein the remote CPU is part of a smart device.

23. The DMF device of claim 0 wherein the communications interface comprises a wired and/or wireless communication interface.

24. The DMF device of claim 0 further comprising computer memory for storing self-test information.

25. A system for performing a self-test for a target analyte, the system comprising:
a. a DMF device of any one of claims 0 through 0; and
b. a self-test application for downloading onto a user device, wherein the self-test application provides a user interface for operating the system and/or the DMF device and instructions for performing a pELISA test for a target analyte.

26. The system of claim 0 wherein the self-test application further comprises:
i. an algorithm for processing digital image data of the pELISA test to produce a colorimetric readout based on a colorimetric change; and
ii. an algorithm for analyzing the colorimetric readout to determine the presence or absence of a target analyte.

27. The system of claim 0 wherein the user interface further comprises a display for presenting the results of the self-test to the user.

28. The system of claim 0 wherein the digital image data comprises image data captured using an image capture device operated by the user.

29. The system of claim 0 wherein the user’s image capture device comprises an on-board camera of the user’s smart device.

30. The system of claim 0 wherein the captured image data is stored in computer memory on the user’s smart device.

31. The system of claim 0 further comprising a communications link for providing a communication path between the DMF device and the user’s smart device.

32. The system of claim 0 further comprising a data storage associated with a networked computer via a network for storing and sharing the self-test information.

33. A method of performing a biological analysis for a target analyte, the method comprising:
a. providing a DMF device of any one of claims 0 through 0;
b. providing a reaction surface and a capture molecule in the one or more reaction chambers or reaction zones in the droplet operations gap of the DMF device;
c. using droplet operations effected by the controller:
i. introducing a sample fluid onto the reaction surface, wherein the sample fluid potentially comprises a target analyte that binds to the capture molecule, forming a target-capture molecule complex immobilized on the reaction surface;
ii. introducing a detection antibody onto the reaction surface, wherein:
1. an enzyme is conjugated to the detection antibody; and/or
2. the enzyme is conjugated to the capture molecule;
iii. introducing a detection solution comprising an enzyme substrate onto the reaction surface, wherein in the presence of a target-capture molecule complex a colorimetric change is produced; and
d. measuring at the one or more detection spots in each of the one or more reaction chambers or reaction zones the colorimetric change in response to the enzyme catalyzed detection of the target analyte.

34. The method of claim 0 wherein the reaction surface comprises a plasmonic nanoparticle immobilized thereon and the capture molecule is suspended in a solution on the reaction surface.

35. The method of claim 0 wherein the reaction surface comprises a plasmonic nanoparticle and a capture molecule immobilized thereon.

36. The method of claim 0 wherein the reaction surface comprises the capture molecule immobilized thereon, and the detection solution further comprises a plasmonic nanoparticle.

37. The method of claim 0 and following wherein the plasmonic nanoparticle comprises a nanosphere, a nanorod, a nanourchin, or a nanostar.

38. The method of claim 0 and following wherein the plasmonic nanoparticle comprises two or more types of plasmonic nanoparticles, thereby increasing the sensitivity and/or range of detection for a target analyte.

39. The method of claim 0 and following wherein the plasmonic nanoparticle comprises a gold nanoparticle.

40. The method of claim 0 wherein the gold nanoparticle comprises a gold nanosphere and/or a gold nanourchin.

41. The method of claim 0 wherein the reaction surface comprises a substrate surface of the DMF device.

42. The method of claim 0 wherein the reaction surface comprises a magnetically responsive bead.

43. The method of claim 0 wherein the capture molecule comprises an antibody.

44. The method of claim 0 wherein the capture molecule comprises an antigen.

45. The method of claim 0 wherein the sample fluid comprises a bodily fluid from a human or an animal.

46. The method of claim 0 wherein the target analyte comprises two or more target analytes.

47. The method of claim 0 wherein the target analyte is a protein.

48. The method of claim 0 wherein the protein is an antibody.

49. The method of claim 0 wherein the antibody is an IgG or IgM antibody.

50. The method of claim 0 wherein the target analyte is a molecule or molecular structure from a virus, a bacterium, or any other pathogen.

51. The method of claim 0 wherein the target analyte comprises a molecule or molecular structure bound to the outer surface of a virus, a bacterium, or any other pathogen.

52. The method of claim 0 wherein the target analyte comprises a molecule or molecular structure that is internal to a virus, a bacterium, or any other pathogen.

53. The method of claim 0 wherein the internal molecule or molecular structure is exposed by disrupting the integrity of the virus, the bacterium, or any other pathogen.

54. The method of claim 0 wherein the detection antibody comprises a primary antibody conjugated to an enzyme.

55. The method of claim 0 wherein the detection antibody comprises a secondary antibody conjugated to an enzyme.

56. The method of claim 0 wherein the enzyme comprises horseradish peroxidase (HRP).

57. The method of claim 0 wherein the enzyme substrate comprises TMB.

58. The method of claim 0 wherein the detection solution further comprises a metal ion precursor.

59. The method of claim 0 wherein the detection solution further comprises a fluorescent probe.

60. The method of claim 0 wherein the colorimetric change comprises a change in the intensity of a color and/or perceivable color hue.

61. The method of claim 0 wherein the colorimetric change is caused by etching of the plasmonic nanoparticle in response to the enzyme catalyzed detection of the target analyte.

62. The method of claim 0 wherein the colorimetric change is caused by aggregation of the plasmonic nanoparticle in response to the enzyme catalyzed detection of the target analyte.

63. The method of claim 0 wherein the colorimetric change is caused by growth of the plasmonic nanoparticle in response to the enzyme catalyzed detection of the target analyte.

64. The method of claim 0 wherein the colorimetric change is caused by quenching and/or unquenching the fluorescence of a fluorescent probe in response to the enzyme catalyzed detection of the target analyte.

65. The method ofclaim 0 wherein measuring the colorimetric change comprises:
i. capturing a digital image of the colorimetric changes at each detection spot of the one or more reaction chambers or reaction zones;
ii. processing the digital image data to produce a colorimetric readout based on the colorimetric change; and
iii. analyzing the colorimetric readout to determine the presence or absence of the target analyte.

66. The method ofclaim 0 wherein processing the digital image data comprises using a color-based detection algorithm to produce the colorimetric readout.

67. The method of claim 0 where in analyzing the colorimetric readout comprises using an algorithm to differentiate a positive or a negative sample based on the colorimetric results.

68. The method of claim 0 further comprising concentrating the target analyte prior to analysis.

69. A method of performing a user conducted self-test for a target analyte, the method comprising:
a. providing the system of claim 25 to a user;
b. downloading the self-test application onto the user’s smart device to initiate and set up the self-testing process;
c. introducing a user sample into one or more sample reservoirs of the DMF device, wherein the pELISA test is automatically performed to test for the presence or absence of the target analyte; and
d. capturing a digital image of the pELISA test results for automated analysis for determining the presence or absence of the target analyte.

70. The method of claim 0 wherein setting up the self-testing process comprises:
i. establishing a communication link between the user’s smart device and the DMF device; and
ii. capturing an image of a QR code provided on the DMF device and collecting any other required test information.

71. The method of claim 0 wherein the user sample comprises a saliva sample.

72. The method of claim 0 further comprising presenting the self-test results to the user.

73. The method of claim 0 further comprising sharing the results of the self-test with a networked computer.

74. The method of claim 0 further comprising stopping the self-testing processes if the user decides that he/she are not ready to continue the testing process.

75. The method of claim 0 further comprising introducing an assay buffer and detection solution into one or more reagent wells of the DMF device.
